(19) United States
(12) Patent Application Publication
(10) Pub. No.: US 2021/0285912 A1
(43) Pub. Date: Sep. 16, 2021

(54) CONFIGURABLE ELECTROCHEMICAL GAS SENSOR

(71) Applicant: InSyte Systems, Inc., Newark, CA (US)

(72) Inventors: Jim Chih-Min Cheng, Fremont, CA (US); Jerome Chandra Bhat, Palo Alto, CA (US); Eric Paul Lee, Mountain View, CA (US); Yifan Jiang, San Leandro, CA (US)

(21) Appl. No.: 17/199,649

(22) Filed: Mar. 12, 2021

Related U.S. Application Data
(60) Provisional application No. 62/988,732, filed on Mar. 12, 2020.

(51) Int. Cl. G01N 27/406; G01N 27/407

(52) U.S. Cl. CPC G01N 27/4065; G01N 27/407

(57) ABSTRACT

An electrochemical gas detector includes an electrochemical cell, a switching circuit, and a drive circuit. The switching circuit is electrically coupled to the electrochemical cell and to the switching circuit. The drive circuit includes a working-electrode terminal, a counter-electrode terminal, and a reference-electrode terminal. The electrochemical cell includes a first electrode, a second electrode, and a third electrode. The switching circuit has a first state in which the first electrode is electrically coupled to the working-electrode terminal, the second electrode is electrically coupled to the counter-electrode terminal, and the third electrode is electrically coupled to the reference-electrode terminal. The switching circuit has a second state in which the first electrode is electrically coupled to the counter-electrode terminal, the second electrode is electrically coupled to the working-electrode terminal, and the third electrode is electrically coupled to the reference-electrode terminal. A second drive circuit can be electrically coupled to the switching circuit.

### CROSS-REFERENCE TO RELATED APPLICATIONS

This application claims priority to U.S. Provisional Application No. 62/988,732, titled “Reconfigurable Environmental Sensor,” filed on Mar. 12, 2020, which is hereby incorporated by reference.

### TECHNICAL FIELD

This application relates generally to electrochemical gas sensors.

### BACKGROUND

Given the changes in the earth's atmosphere, precipitated by industrialization and natural sources, as well as the dramatically increasing number of household and urban pollution sources, the need for accurate and continuous air quality monitoring has become necessary to both identify the sources and warn consumers of impending danger. Tantamount to making real-time monitoring and exposure assessment a reality is the ability to deliver, low cost, small form factor, and low power devices which can be integrated into the broadest range of platforms and applications.

There are multiple methods of sensing distinct low-density materials such as gasses. Common methods include nondispersive infrared spectroscopy, metal oxide sensors, chemiresistors, and electrochemical sensors. One drawback with conventional electrochemical sensors is that they have a fixed configuration in which each electrode has a dedicated function. For example, one electrode is set as the working electrode, another electrode is set as the counter electrode, and another electrode is set as the reference electrode. It would be desirable to be able to switch electrode configuration on the sensor, which would have many advantages from selectivity improvement to sensor health monitoring.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is directed to an electrochemical gas detector. The electrochemical gas detector comprises an electrochemical cell, a switching circuit, and a drive circuit. The electrochemical cell comprises a first electrode; a second electrode; a third electrode; and an electrolyte in contact with the first, second, and third electrodes. The switching circuit is electrically coupled to the electrochemical cell. The drive circuit is electrically coupled to the switching circuit, the drive circuit having a working-electrode terminal, a counter-electrode terminal, and a reference-electrode terminal. The switching circuit has a first state in which the first electrode is electrically coupled to the working-electrode terminal, the second electrode is electrically coupled to the counter-electrode terminal, and the third electrode is electrically coupled to the reference-electrode terminal. The switching circuit has a second state in which the first electrode is electrically coupled to the counter-electrode terminal, the second electrode is electrically coupled to the working-electrode terminal, and the third electrode is electrically coupled to the reference-electrode terminal.

In one or more embodiments, the drive circuit comprises a galvanostat or a potentiostat. In one or more embodiments, the drive circuit further comprises a voltmeter, a frequency analyzer, a function generator, an oscilloscope, and/or a network analyzer.

In one or more embodiments, the electrochemical cell comprises a housing, the first, second, and third electrodes disposed in the housing, a first vent hole is defined in the housing to expose a portion of the first electrode, and a second vent hole is defined in the housing to expose a portion of the second electrode. In one or more embodiments, the detector further comprises a gas-selective filter disposed on or in the first vent hole. In one or more embodiments, the gas-selective filter is configured to prevent first gases from passing through the gas-selective filter, and the first and second electrodes comprise a same catalyst that is sensitive to multiple gases including the first gases. In one or more embodiments, the first gases comprise organic gases

In one or more embodiments, the gas-selective filter is a first gas-selective filter and the sensor further comprises a second gas-selective filter disposed on or in the second vent hole. In one or more embodiments, the first and second gas-selective filters are configured to filter different gasses. In one or more embodiments, the first gas-selective filter is configured to only allow carbon monoxide and nitrogen dioxide to pass through the first gas-selective filter, the second gas-selective filter is configured to only allow carbon monoxide to pass through the second gas-selective filter, when the switching circuit is in the first state, the sensor measures a total concentration of carbon monoxide and nitrogen dioxide in an environment of the sensor, and when the switching circuit is in the second state, the sensor measures a concentration of carbon monoxide in the environment. In one or more embodiments, the first and second electrodes comprise different catalysts.

In one or more embodiments, the first and second electrodes comprise different catalysts. In one or more embodiments, the detector further comprises a gas-selective filter disposed on or in the first vent hole.

In one or more embodiments, the drive circuit is a first drive circuit, and the working-electrode terminal, the counter-electrode terminal, and the reference-electrode terminal are a first working-electrode terminal, a first counter-electrode terminal, and a first reference-electrode terminal, respectively. The detector further comprises a second drive circuit electrically coupled to the switching circuit, the second drive circuit having a second working-electrode terminal, a second counter-electrode terminal, and a second reference-electrode terminal. The switching circuit has a third state in which the first electrode is electrically coupled to the second working-electrode terminal, the second electrode is electrically coupled to the second counter-electrode terminal, and the third electrode is electrically coupled to the second reference-electrode terminal. In one or more embodiments, when the switching circuit is in the first state, the second drive circuit is electrically decoupled from the electrochemical cell; when the switching circuit is in the second state, the second drive circuit is electrically decoupled from the electrochemical cell; and when the switching circuit is in the third state, the first drive circuit is electrically decoupled from the electrochemical cell.

Another aspect of the invention is directed to an electrochemical gas detector. The detector comprises an electrochemical cell, a switching circuit, and a drive circuit. The electrochemical cell comprises a first electrode; a second electrode; a third electrode; a fourth electrode; and an electrolyte in contact with the first, second, third, and fourth electrodes. The switching circuit is electrically coupled to the electrochemical cell. The drive circuit is electrically coupled to the switching circuit, the drive circuit having a working-electrode terminal, a counter-electrode terminal, and a reference-electrode terminal. The switching circuit has a first state in which the first electrode is electrically coupled to the working-electrode terminal, the second electrode is electrically coupled to the counter-electrode terminal, and the third electrode is electrically coupled to the reference-electrode terminal. The switching circuit has a second state in which the first electrode is electrically coupled to the working-electrode terminal, the second electrode is electrically coupled to the counter-electrode terminal, and the fourth electrode is electrically coupled to the reference-electrode terminal.

In one or more embodiments, the electrochemical cell comprises a housing, the first, second, third, and fourth electrodes disposed in the housing, a first vent hole is defined in the housing to expose a portion of the first electrode, and a second vent hole is defined in the housing to expose a portion of the second electrode. In one or more embodiments, the switching circuit has a third state in which the second electrode is electrically coupled to the working-electrode terminal, the first electrode is electrically coupled to the counter-electrode terminal, and the third electrode is electrically coupled to the reference-electrode terminal.

In one or more embodiments, the first and second electrodes comprise different catalysts. In one or more embodiments, the detector further comprises a gas-selective filter disposed on or in the first vent hole. In one or more embodiments, the gas-selective filter is configured to prevent first gases from passing through the gas-selective filter, and the first and second electrodes comprise a same catalyst that is sensitive to multiple gases including the first gases.

In one or more embodiments, the gas-selective filter is a first gas-selective filter and the sensor further comprises a second gas-selective filter disposed on or in the second vent hole. In one or more embodiments, the first and second gas-selective filters are configured to filter different gasses. In one or more embodiments, the first gas-selective filter is configured to only allow carbon monoxide and nitrogen dioxide to pass through the first gas-selective filter, the second gas-selective filter is configured to only allow carbon monoxide to pass through the second gas-selective filter, when the switching circuit is in the first state, the sensor measures a total concentration of carbon monoxide and nitrogen dioxide in an environment of the sensor, and when the switching circuit is in the second state, the sensor measures a concentration of carbon monoxide in the environment.

In one or more embodiments, the switching circuit has a fourth state in which the second electrode is electrically coupled to the working-electrode terminal, the first electrode is electrically coupled to the counter-electrode terminal, and the fourth electrode is electrically coupled to the reference-electrode terminal. In one or more embodiments, the drive circuit comprises a galvanostat or a potentiostat.

In one or more embodiments, the drive circuit further comprises a voltmeter, a frequency analyzer, a function generator, an oscilloscope, and/or a network analyzer.

Yet another aspect of the invention is directed to an electrochemical gas detector. The detector comprises an electrochemical cell, a first drive circuit, and a second drive circuit. The electrochemical cell comprises a first electrode; a second electrode; a first reference electrode; a second reference electrode; and an electrolyte in contact with the first, second, first reference, and second reference electrodes. The first drive circuit is electrically coupled to the first electrode, the second electrode, and the first reference electrode. The second drive circuit is electrically coupled to the first electrode, the second electrode, and the second reference electrode.

In one or more embodiments, the first drive circuit and the second drive circuit are configured to operate at different frequencies. In one or more embodiments, an impedance between the first reference electrode and the first electrode is different than an impedance between the second reference electrode and the first electrode. In one or more embodiments, an impedance between the first reference electrode and the second electrode is different than an impedance between the second reference electrode and the second electrode.

In one or more embodiments, the detector further comprises a switching circuit electrically coupled to the electrochemical cell, the first drive circuit, and the second drive circuit, wherein: the first drive circuit includes a first working-electrode terminal, a first counter-electrode terminal, and a first reference-electrode terminal; the second drive circuit includes a second working-electrode terminal, a second counter-electrode terminal, and a second reference-electrode terminal; the switching circuit has a first state in which the first electrode is electrically coupled to the first working-electrode terminal, the second electrode is electrically coupled to the first counter-electrode terminal, and the first reference electrode is electrically coupled to the first reference-electrode terminal; and the switching circuit has a second state in which the first electrode is electrically coupled to the first counter-electrode terminal, the second electrode is electrically coupled to the first working-electrode terminal, and the first reference electrode is electrically coupled to the first reference-electrode terminal. In one or more embodiments, the switching circuit has a third state in which the first electrode is electrically coupled to the second working-electrode terminal, the second electrode is electrically coupled to the second counter-electrode terminal, and the second reference electrode is electrically coupled to the second reference-electrode terminal; and the switching circuit has a fourth state in which the first electrode is electrically coupled to the second counter-electrode terminal, the second electrode is electrically coupled to the second working-electrode terminal, and the second reference electrode is electrically coupled to the second reference-electrode terminal.

Another aspect of the invention is directed to a method of operating an electrochemical gas sensor, comprising: electrically coupling a switching circuit to an electrochemical cell that comprises a first electrode, a second electrode, and a third electrode; electrically coupling a drive circuit to the switching circuit, the drive circuit including a working-electrode terminal, a counter-electrode terminal, and a reference-electrode terminal; placing the switching circuit in a first state in which the first electrode is electrically coupled to the working-electrode terminal, the second electrode is electrically coupled to the counter-electrode terminal, and the third electrode is electrically coupled to the reference-electrode terminal; and placing the switching circuit in a second state in which the second electrode is electrically coupled to the working-electrode terminal, the first electrode is electrically coupled to the counter-electrode terminal, and the third electrode is electrically coupled to the reference-electrode terminal.

In one or more embodiments, the method further comprises: when the switching circuit is in the first state, determining, with a computer in electrical communication with the drive circuit, a concentration of a first gas in an environment of the electrochemical gas sensor, the computer including a microprocessor and memory that is operatively coupled to the microprocessor; and when the switching circuit is in the second state, determining, with the computer, a concentration of the second gas in the environment.

In one or more embodiments, the method further comprises: filtering ambient gases with a first filter that only allows one or more first gas(es) to pass through to the first electrode; and when the switching circuit is in the first state, determining, with a computer in electrical communication with the drive circuit, a total concentration of the one or more first gas(es) in an environment of the electrochemical gas sensor, the computer including a microprocessor and memory that is operatively coupled to the microprocessor. In one or more embodiments, the method further comprises: when the switching circuit is in the second state, determining with the computer, a state of the electrochemical cell; and with the computer, adjusting a determination of the total concentration of the one or more first gas(es) based, at least in part, on the state of the electrochemical cell.

In one or more embodiments, the method further comprises: filtering ambient gases with a first filter that only allows first and second gases to pass through to the first electrode; filtering the ambient gases with a second filter that only allows the first gas to pass through to the second electrode; when the switching circuit is in the first state, determining, with a computer in electrical communication with the drive circuit, a total concentration of the first and second gases in an environment of the electrochemical gas sensor, the computer including a microprocessor and memory that is operatively coupled to the microprocessor; and when the switching circuit is in the second state, determining, with the computer, a concentration of the first gas in the environment.

In one or more embodiments, the method further comprises determining, with the computer, a concentration of the second gas in the environment based on the total concentration of the first and second gases and the concentration of the first gas. In one or more embodiments, the first gas comprises nitrogen dioxide and the second gas comprises carbon monoxide.

In one or more embodiments, the method further comprises: filtering ambient gases with a first filter that only allows inorganic gases to pass through to the first electrode; when the switching circuit is in the first state, determining, with a computer in electrical communication with the drive circuit, a concentration of the inorganic gases in an environment of the electrochemical gas sensor, the computer including a microprocessor and memory that is operatively coupled to the microprocessor; when the switching circuit is in the second state, determining, with the computer, a total concentration of the organic and inorganic gases in the environment; and determining, with the computer, a concentration of the organic gases in the environment based on the concentration of the inorganic gases and the total concentration of the organic and inorganic gases.

In one or more embodiments, the method further comprises: filtering ambient gases with a first filter that only allows organic gases to pass through to the first electrode; when the switching circuit is in the first state, determining, with a computer in electrical communication with the drive circuit, a concentration of the organic gases in an environment of the electrochemical gas sensor, the computer including a microprocessor and memory that is operatively coupled to the microprocessor; when the switching circuit is in the second state, determining, with the computer, a total concentration of the organic and inorganic gases in the environment; and determining, with the computer, a concentration of the inorganic gases in the environment based on the concentration of the organic gases and the total concentration of the organic and inorganic gases.

Yet another aspect of the invention is directed to a method of sensing a gas, comprising: electrically coupling a first drive circuit to a first group of electrodes in an electrochemical cell; electrically coupling a second drive circuit to a second group of electrodes in the electrochemical cell; using the first drive circuit and the first group of electrodes to take a first gas measurement of an environment of the electrochemical cell; and using the second drive circuit and the second group of electrodes to take a second gas measurement of the environment.

In one or more embodiments, the first and second groups of electrodes are the same. In one or more embodiments, the first group of electrodes is different than the second group of electrodes. In one or more embodiments, the first and second group electrodes do not include a common electrode.

In one or more embodiments, the method further comprises: operating the first drive circuit at a first frequency; and operating the second drive circuit at a second frequency that is lower than the first frequency. In one or more embodiments, the method further comprises: operating the first drive circuit at a first frequency range that includes the first frequency; and performing electrochemical impedance spectroscopy over the first frequency range. In one or more embodiments, the first frequency range comprises about 1 kHz to about 1 MHz, and the second frequency range comprises about 0 kHz to about 1 kHz.

In one or more embodiments, the method further comprises determining, in a computer comprising a microprocessor, a composition of the gas using the first gas measurement and/or the second gas measurement. In one or more embodiments, the method further comprises: receiving, at the computer, environment data from an external data source; using the environment data to determine, in the computer, the composition of the gas and/or a concentration of the gas in the environment; and generating an output signal, in the computer, that corresponds to the composition of the gas and/or the concentration of the gas. In one or more embodiments, the environment data comprises a temperature of the environment, a relative humidity of the environment, an atmospheric pressure of the environment, and/or geolocation data of the computer.

Another aspect of the invention is directed to a method of sensing a gas, comprising: electrically coupling a first drive circuit to a group of electrodes in an electrochemical cell; using the first drive circuit and the group of electrodes to take a gas measurement of an environment of the electrochemical cell; electrically disconnecting the group of electrodes from the first drive circuit; electrically coupling a second drive circuit to the group of electrodes; using the second drive circuit and the group of electrodes to determine a state of the electrochemical cell; and determining, with a computer in electrical communication with the first and second drive circuits, a composition and/or a concentration of the gas in the environment of the electrochemical cell using the gas measurement and the state of the electrochemical cell.

In one or more embodiments, the state of the electrochemical cell comprises an impedance between two electrodes in the group of electrodes.

### BRIEF DESCRIPTION OF THE DRAWINGS

For a fuller understanding of the nature and advantages of the present concepts, reference is made to the detailed description of preferred embodiments and the accompanying drawings.

FIG. 1 is a block diagram of an electrochemical gas sensor apparatus in a first state according to an embodiment.

FIG. 2 is a block diagram of the electrochemical gas sensor apparatus illustrated in FIG. 1 in a second state according to an embodiment.

FIG. 3 is a block diagram of the electrochemical gas sensor apparatus illustrated in FIG. 1 in a third state according to an embodiment.

FIG. 4 is a block diagram of the electrochemical gas sensor apparatus illustrated in FIG. 1 in a fourth state according to an embodiment.

FIG. 5 is a block diagram of the electrochemical cell illustrated in FIGS. 1-4 according to an embodiment.

FIG. 6 is a block diagram of the electrochemical cell illustrated in FIGS. 1-4 according to another embodiment.

FIG. 7 is a block diagram of the electrochemical cell illustrated in FIGS. 1-4 according to another embodiment.

FIG. 8 is a block diagram of an electrochemical gas sensor apparatus in a first state according to another embodiment.

FIG. 9 is a block diagram of the electrochemical gas sensor apparatus illustrated in FIG. 8 in a second state according to an embodiment.

FIG. 10 is a block diagram of the electrochemical cell illustrated in FIGS. 8 and 9 according to an embodiment.

FIG. 11 is a block diagram of an electrochemical gas sensor apparatus according to another embodiment.

FIG. 12 is a flow chart of a method of operating an electrochemical gas sensor according to an embodiment.

FIG. 13 is a flow chart of a method of sensing a gas according to an embodiment.

FIG. 14 is a flow chart of a method of sensing a gas according to an embodiment.

### DETAILED DESCRIPTION

An electrochemical gas sensor apparatus includes an electrochemical cell, a switching circuit, and a drive and sense circuit (e.g., a drive and sense circuit). The switching circuit is electrically coupled to the electrochemical cell and to the drive circuit. The switching circuit has multiple states in which the drive circuit is electrically coupled differently to the electrodes in the electrochemical cell. For example, in a first state of the switching circuit, a first electrode can be electrically coupled to a working-electrode terminal in the drive circuit, a second electrode can be electrically coupled to a counter-electrode terminal in the drive circuit, and a third electrode optionally can be electrically coupled to a reference-electrode terminal in the drive circuit. In a second state of the switching circuit, the second electrode can be electrically coupled to a driving-electrode terminal in the drive circuit, the first electrode can be electrically coupled to a counter-electrode terminal in the drive circuit, and the third electrode optionally can be electrically coupled to a reference-electrode terminal in the drive circuit. Thus, in the first state the first electrode functions as the working electrode, the second electrode functions as the counter electrode, and the third electrode optionally functions as the reference electrode, while in the second state the second electrode functions as the working electrode, the first electrode functions as the counter electrode, and the third electrode optionally functions as the reference electrode. Additional states and configurations are disclosed.

A gas-selective filter can be disposed on or in the vent hole for an electrode. The gas-selective filter can limit or restrict the gas(es) that can flow through the gas-selective filter to reach the electrode. One or more additional gas-selective filters can be disposed on in or the vent hole(s) of one or more additional electrodes. Each gas-selective filter can be the same or different than the other gas-selective filters, for example to limit or restrict different gases. This filter may also serve the function of “zeroing” the gas stream to remove all gas components except small gases such as oxygen and nitrogen for the sensor. Additionally, variants of filter may serve to protect the internal components of the sensor from condensing humidity (e.g., providing water resistance or waterproofing).

Additionally or alternatively, the drive circuit can include multiple drive circuits. Each drive circuit is electrically coupled to a respective group of electrodes, which can be the same, different, or overlapping. This can allow different electrodes to function simultaneously as working electrodes to sense different gas concentrations simultaneously. Alternatively, the first and second drive circuits can operate at different frequencies, for example while connected to the same group of electrodes. The first drive circuit can operate at a relatively low frequency to provide a stable, low-noise measurement while the second drive circuit can operate at a high frequency to provide measurements that are the same or similar to electrochemical impedance spectroscopy and/or voltammetry. In another embodiment, the first drive circuit can include a first digital-to-analog converter (DAC) and the second drive circuit can include a second DAC. The first DAC can be used to perform gross-stepping voltammetry and the second DAC can be used to perform voltammetry with a fine resolution, which can allow for large-range voltammetry with a fine resolution.

The first and second drive circuits can operate simultaneously or iteratively. When the first and second drive circuits are coupled to at least one common electrode, the first and second drive circuits are preferably driven iteratively to electrically isolate the first and second drive circuits and the respective electrodes. When the first and second drive circuits are not coupled to any common electrodes, the first and second drive circuits can be driven simultaneously or iteratively.

The interchangeability of working and counter electrodes, as described herein, can provide a first degree of selectivity to the sensor to different gasses. The use of different gas-selective filters can provide a second degree of selectivity to the sensor to different gasses. The use of electrochemical impedance spectroscopy and/or voltammetry can provide a third degree of selectivity to the sensor to different gasses. Having different catalysts on the different electrodes would provide a fourth degree of selectivity on the different electrodes, especially with the interchangeability. All four mechanisms may be used together so as to maximize selectivity of the sensor to different gasses. Additional details regarding selectivity of electrochemical sensors and/or other aspects of this disclosure are disclosed in U.S. Patent Application Publication No. 2018/0231494, titled Electrochemical Gas Sensor System With Improved Accuracy and Speed,” which is hereby incorporated by reference.

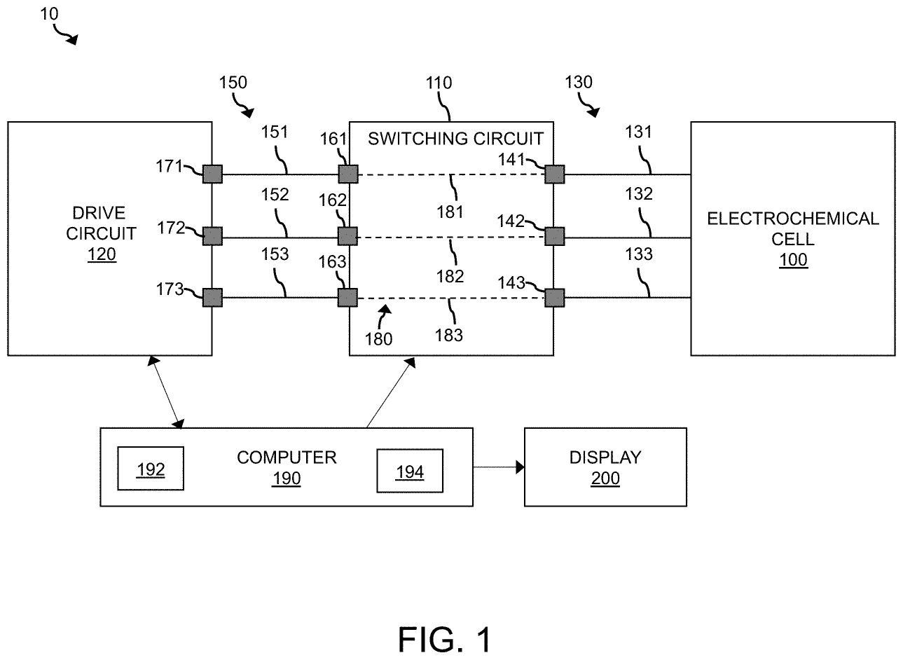

FIG. 1 is a block diagram of an electrochemical gas sensor apparatus 10 according to an embodiment. The apparatus 10 includes an electrochemical cell 100, a switching circuit 110, and a drive circuit 120. The electrochemical cell 100 includes multiple electrodes that are contained in a housing. The housing includes at least one vent hole that exposes at least one respective electrode to the ambient gas of the environment. A gas-selective filter can be disposed on or in any of the vent hole(s) to restrict the gas(es) that can pass through to the respective electrode(s). In a preferred embodiment, the electrochemical cell 100 includes 2, 3, or 4 electrodes. In other embodiments, the electrochemical cell 100 can include an array of electrodes (e.g., at least 2 electrodes (e.g., N electrodes) such as 2-10 electrodes).

The electrochemical cell 100 is electrically coupled to the switching circuit 110 using a plurality of electrical connections 130. For example, a first electrical connection 131 can electrically couple the switching circuit 110 (e.g., a first electrochemical (EC)-side terminal 141 in the switching circuit 110) to a first electrode in the electrochemical cell 100, a second electrical connection 132 can electrically couple the switching circuit 110 (e.g., a second EC-side terminal 142 in the switching circuit 110) to a second electrode in the electrochemical cell 100, and a third electrical connection 133 can electrically couple the switching circuit 110 (e.g., a third EC-side terminal 143 in the switching circuit 110) to a third electrode in the electrochemical cell 100. There can be additional or fewer electrical connections 130 in other embodiments. In one example, when the electrochemical cell 100 includes N electrodes, there can be N electrical connections between the N electrodes and the switching circuit 110. Each electrical connection 130 can comprise a wire, a conductive conduit, or another electrical connection.

The switching circuit 110 is electrically coupled to the drive circuit 120 using a plurality of electrical connections 150. For example, a first electrical connection 151 can electrically couple the switching circuit 110 (e.g., a first drive-circuit (DC)-side terminal 161 in the switching circuit 110) to a working electrode-terminal 170 in the drive circuit 120. A second electrical connection 152 can electrically couple the switching circuit 110 (e.g., a second DC-side terminal 162 in the switching circuit 110) to a counter-electrode terminal 171 in the drive circuit 120. A third electrical connection 153 can electrically couple the switching circuit 110 (e.g., a third DC-side terminal 163 in the switching circuit 110) to an optional reference-electrode terminal 173 in the drive circuit 120. There can be additional or fewer electrical connections 150 in other embodiments.

The switching circuit 110 has different states in which the EC-side terminals 141-143 are electrically coupled to the DC-side terminals 161-163. The switching circuit 110 can remain in a given state for a relatively long or short period of time. An example of a relatively long time period is about 40 mins to about 60 mins or longer. An example of a relatively short time period is about 1 second to about 5 minutes. In some embodiments, the switching circuit 110 changes states frequently and/or can alternate between two or more states repeatedly. For example, the switching circuit 110 can repeatedly transition between first and second states at a predetermined frequency.

In one example, the switching circuit 110 can be in the first state for about 1 second to about 5 minutes and in the second state for about 5 seconds to about 40 minutes. In another example, the switching circuit 110 can be in the first state for about 1 second to about 5 minutes and in the second state for about 1 second to about 5 minutes. In yet another example, the switching circuit 110 can be in the first state for about 5 seconds to about 40 minutes and in the second state for about 5 seconds to about 40 minutes. Additional time variations can also be used.

FIG. 1 illustrates the first state of the switching circuit 110 in which the first EC-side terminal 141 is electrically coupled to the first DC-side terminal 161, the second EC-side terminal 142 is electrically coupled to the second DC-side terminal 162, and the third EC-side terminal 143 is electrically coupled to the third DC-side terminal 163. Thus, in the first state the first electrode in the electrochemical cell 100 is electrically coupled to the working-electrode terminal 171 in the drive circuit 120, the second electrode in the electrochemical cell 100 is electrically coupled to the counter-electrode terminal 172 in the drive circuit 120, and the third electrode in the electrochemical cell 100 is electrically coupled to the optional reference-electrode terminal 173 in the drive circuit 120.

The EC-side terminals 141-143 and the DC-side terminals 161-163 can be electrically coupled using switching electrical connections 181-183 (in general, switching connections 180), respectively. The switching circuit 110 can include additional or fewer switching electrical connections 180 in other embodiments. The switching circuit 110 can comprise a circuit board, a solid-state circuit, an integrated circuit, or another circuit. The switching circuit 110 can include a plurality of physical switches that alter the electrical path between the EC-side terminals 141-143 and the DC-side terminals 161-163.

The drive circuit 120 includes circuitry for driving the electrodes in the electrochemical cell 100 and for sensing a current passing between at least two electrodes, an electrical potential between at least any two electrodes, an electrical potential between an electrode and circuit defined potential external of the electrochemical cell 100, and/or a resistance or impedance between at least any two electrodes, which can indicate the presence or concentration of one or more gases or otherwise characterize, or be indicative of, the state of the electrochemical cell 100. The drive circuit 120 can comprise a galvanostat or a potentiostat in some embodiments. The drive circuit 120 can also comprise a voltmeter, a frequency analyzer, a function generator, an oscilloscope, and/or a network analyzer. In addition, the drive circuit 120 can comprise of one or more DACs and/or multiple resistance ladders to ensure a wide potential range can be scanned with fine resolution.

The drive circuit 120 and/or the switching circuit 110 can be in electrical communication with a computer 190. The computer 190 includes a microprocessor 192 and memory 194 which can include volatile and non-volatile memory. The microprocessor 192 is operatively coupled to the memory 194. The microprocessor 192 can be configured (e.g., through computer-readable instructions (e.g., software) stored in memory 194) to perform one or more functions. For example, the microprocessor 192 can be configured to receive electrical signals from the drive circuit 120 that correspond to the current and/or resistance of the electrochemical cell 100 (e.g., of the electrodes that are electrically coupled to the drive circuit 120) as a function of time. A change in current and/or resistance can correspond to the presence of one or more detected gases and the magnitude in the change in current and/or resistance can correspond to the concentration of the detected gas(es) in the environment. The memory 194 can include a database, lookup tables, and/or mathematical models to determine the concentration of the detected gas(es) as a function of the change in current and/or resistance. The microprocessor 192 can be configured to determine the concentration of the detected gas(es) using the database, lookup tables, and/or mathematical models in the memory 194. In addition, the microprocessor 192 can be configured to generate an output signal that causes a display 200 to graphically display the concentration of the detected gas(es).

The computer 190 can additionally optionally be configured to access data from external sensors or other sources of information which it can further use to more accurately differentiate the gas types and concentrations which are being detected by the electrochemical cell 100 based on the environment in which the electrochemical cell 100 is being used (“context-awareness”). For example, the computer can leverage ambient temperature, relative humidity, and/or atmospheric pressure data to further refine gas concentration calculations. The computer 190 can also leverage geo-location data to understand the likely gasses to which it is being exposed, hence more quickly and accurately identify the gasses being measured by the sensor. For example, formaldehyde would be a gas which is rarely present in outdoor settings, but frequently present in indoor settlings. Additional details regarding context-awareness of the apparatus 10 and/or other aspects of this disclosure are described in U.S. Pat. No. 10,732,141 (“the '141 patent”), titled “Electrochemical Gas Sensor with Varying Bias Voltage and Environmental Compensation,” which is hereby incorporated by reference.

The microprocessor 192 may also have encryption or may be programmed with encryption to limit access to the data from external sources thus securing the data only to authorized programs/apps. This is preferable from a software pipeline perspective for health applications for example where data security can be important. The computer 190 in this case can output final user-readable data (e.g., gas concentration, type, etc.) making this system a fully integrated, secure gas detection system, not just a standalone gas sensor.

In some embodiments, the microprocessor 192 can be configured to send a control signal to the drive circuit 120 to set the frequency of the drive circuit 120 which can correspond to the sampling frequency of the electrochemical gas sensor apparatus 10. In some embodiments, the microprocessor 192 can send the control signal to the drive circuit 120 in response to a user input signal, which the computer 190 can receive via a wired or a wireless connection.

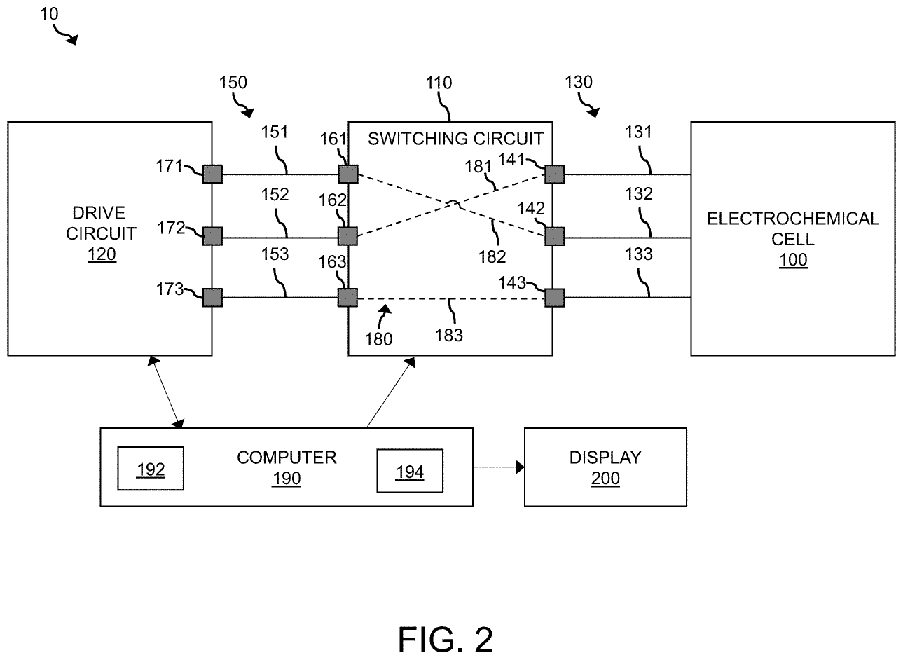

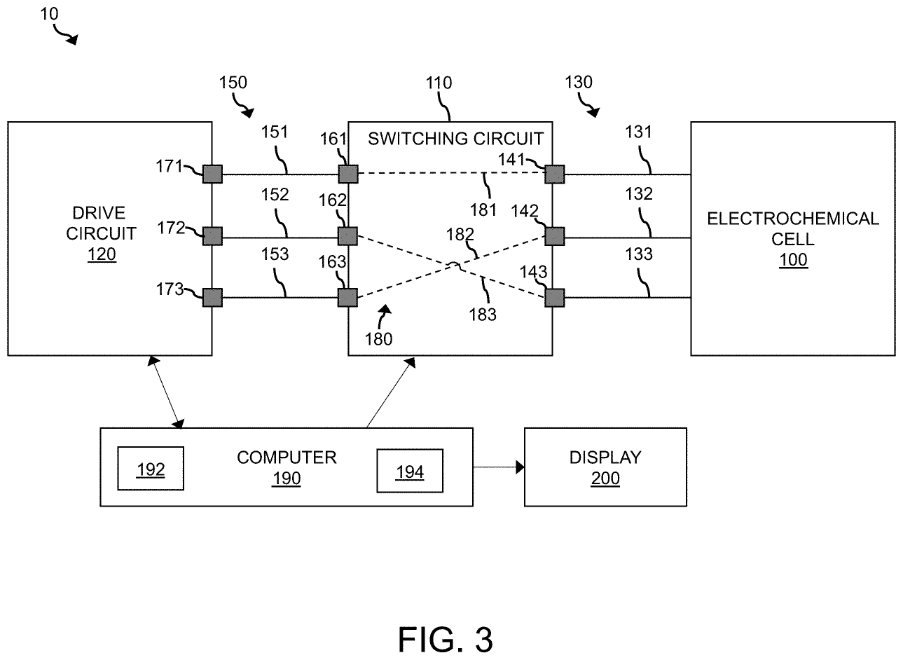

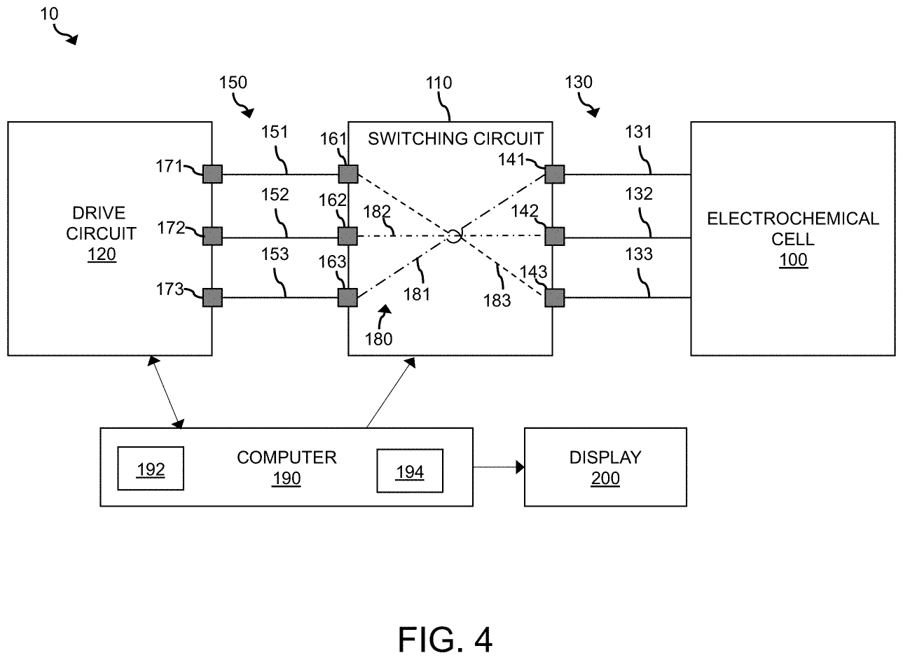

In another example, the microprocessor 192 can be configured to send a control signal to the switching circuit 110 that causes the switching circuit 110 to be configured in a desired state. For example, the control signal can cause the switching circuit 110 to transition to the first state (e.g., as illustrated in FIG. 1), the second state (e.g., as illustrated in FIG. 2), the third state (e.g., as illustrated in FIG. 3), and/or the fourth state (e.g., as illustrated in FIG. 4). The control signal can cause one or more switches (e.g., physical switches, solid-state switches, or other switches) to change state to alter the switching connections 180 between the EC-side terminals 141-143 and the DC-side terminals 161-163. In some embodiments, the microprocessor 192 can send the control signal to the switching circuit 110 in response to a user input signal, which the computer 190 can receive via a wired or a wireless connection.

Two or more components of the electrochemical gas sensor apparatus 10 can be disposed or combined in a single housing. For example, the switching circuit 110 and the drive circuit 120 can be disposed or combined in a single housing. Additionally or alternatively, the computer 190 and the drive circuit 120 can be disposed or combined in a single housing. In another example, the electrochemical cell 100, the switching circuit 110, and the drive circuit 120 can be combined or disposed in a single housing. In yet another example, the electrochemical cell 100, the switching circuit 110, the drive circuit 120, the computer 190, and/or the display 200 can be combined or disposed in a single housing.

FIG. 2 is a block diagram of the electrochemical gas sensor apparatus 10 when the switching circuit 110 is in a second state according to an embodiment. In the second state, the first EC-side terminal 141 is electrically coupled to the second DC-side terminal 162, the second EC-side terminal 142 is electrically coupled to the first DC-side terminal 161, and the third EC-side terminal 143 is electrically coupled to the third DC-side terminal 163. Thus, in the second state the first electrode in the electrochemical cell 100 is electrically coupled to the counter-electrode terminal 172 in the drive circuit 120, the second electrode in the electrochemical cell 100 is electrically coupled to the working-electrode terminal 171 in the drive circuit 120, and the third electrode in the electrochemical cell 100 is electrically coupled to the optional reference-electrode terminal 173 in the drive circuit 120.

FIG. 3 is a block diagram of the electrochemical gas sensor apparatus 10 when the switching circuit 110 is in a third state according to an embodiment. In the third state, the first EC-side terminal 141 is electrically coupled to the first DC-side terminal 161, the second EC-side terminal 142 is electrically coupled to the third DC-side terminal 163, and the third EC-side terminal 143 is electrically coupled to the second DC-side terminal 162. Thus, in the third state the first electrode in the electrochemical cell 100 is electrically coupled to the working-electrode terminal 171 in the drive circuit 120, the second electrode in the electrochemical cell 100 is electrically coupled to the optional reference-electrode terminal 173 in the drive circuit 120, and the third electrode in the electrochemical cell 100 is electrically coupled to the counter-electrode terminal 172 in the drive circuit 120.

FIG. 4 is a block diagram of the electrochemical gas sensor apparatus 10 when the switching circuit 110 is in a fourth state according to an embodiment. In the fourth state, the first EC-side terminal 141 is electrically coupled to the third DC-side terminal 163, the second EC-side terminal 142 is electrically coupled to the second DC-side terminal 162, and the third EC-side terminal 143 is electrically coupled to the first DC-side terminal 161. Thus, in the third state the first electrode in the electrochemical cell 100 is electrically coupled to the optional reference-electrode terminal 173 in the drive circuit 120, the second electrode in the electrochemical cell 100 is electrically coupled to the counter-electrode terminal 172 in the drive circuit 120, and the third electrode in the electrochemical cell 100 is electrically coupled to the working-electrode terminal 171 in the drive circuit 120.

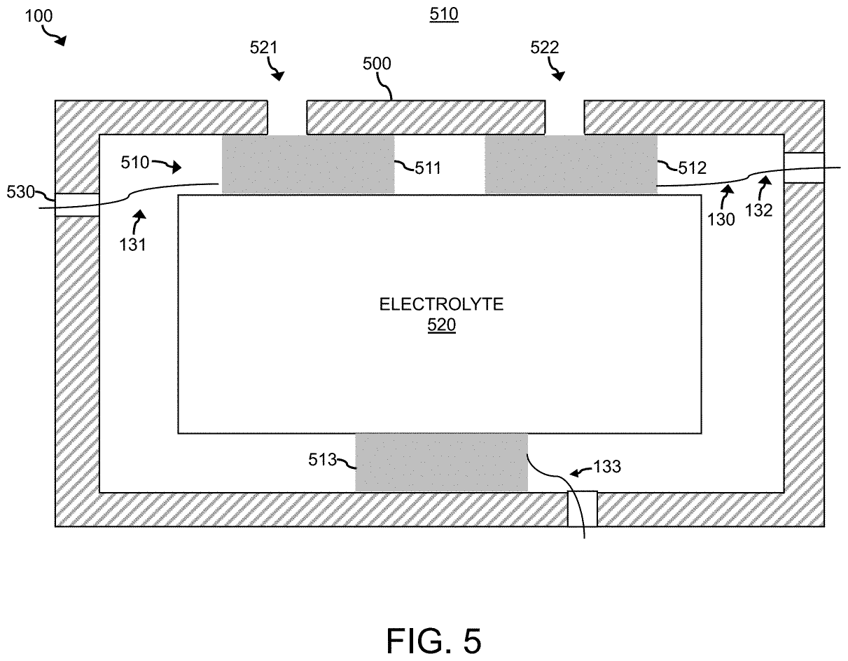

FIG. 5 is a block diagram of the electrochemical cell 100 illustrated in FIGS. 1-4 according to an embodiment. The electrochemical cell 100 includes a housing 500 in which a plurality of (e.g., at least two) electrodes 510 are disposed. The electrodes 510 include a first electrode 511, a second electrode 512, and an optional third electrode 513. A portion the first and second electrodes 511, 512 is exposed to ambient gas in the environment 510 through respective first and second vent holes 521, 522 formed in the housing 500. The housing 500 creates a fluid-tight seal around the electrodes 510 such that the only fluid path to the electrodes 510 is through the first and second vent holes 521, 522. Gaskets or seals can be used in the housing 500 (e.g., between the housing 500 and the first and second electrodes 511, 512) to create or improve a fluid-tight seal (e.g., a gas-tight seal). The housing 500 can include (e.g., can be made of) any material having sufficient chemical inertness to the electrolyte 520, and being able to be formed such that electrical conduits can be passed through them. Example materials include, but are not limited to: ceramics, glasses, silicon, epoxies, and/or polymers. Housings made from these materials can be formed with electrical conduits comprising, but not limited to, metals such as tungsten, nickel, copper, and/or other metals.

The electrodes 510 can comprise the same or different active materials or catalysts. In one embodiment, the first and second electrodes 511, 512 comprise different catalysts. For example, the first electrode 511 can comprise a first catalyst that can detect (e.g., that can catalyze a reaction with) one or more first gases, and the second electrode 512 can comprise a second catalyst that can detect (e.g., that can catalyze a reaction with) one or more second gases. The optional third electrode 513 can comprise the first catalyst, the second catalyst, a different catalyst than the first and second catalysts, or no catalyst.

In another embodiment, the first and second electrodes 511, 512 comprise the same catalyst(s). In one example, the first and second electrodes 511, 512 comprise identical or substantially (e.g., within manufacturing limits such as within 0.1%) identical catalysts. The composition or weight percentage of the catalysts in the first and second electrodes 511, 512 can be the same or substantially the same (e.g., within manufacturing limits such as within 0.1%). In a specific embodiment, the first and second electrodes 511, 512 can be identical or substantially identical. The optional third electrode 513 can comprise the same catalyst(s) as the first and second electrodes 511, 512, a different catalyst than the first and second electrodes 511, 512, or no catalyst. In a preferred embodiment, the optional third electrode 513 functions as a reference electrode.

One of more of the electrodes 510 can comprise a porous material and a catalyst. The porous material can comprise carbon paper, carbon cloth, and/or any other porous, electrically conducting matrix. The catalyst can comprise platinum, palladium, ruthenium, rhodium, gold, silver, copper, cobalt, nickel, iron, vanadium, iridium, osmium, and/or other suitable transition metal(s) and alloy(s) thereof. Additionally or alternatively, the catalyst can comprise an aluminosilicate, alumina, boron nitrides, and/or mixtures thereof. Additionally or alternatively, one of more of the electrodes 510 can comprise a non-porous material and a catalyst. Non-porous, but gas permeable, materials include polymers such as PDMS (polydimethylsiloxane) and polyacetylenes. Non-porous electrode support materials which are not gas permeable can be of many materials, such as, but not limited to, ceramics, glasses, silicon, epoxies, and/or polymers. Additionally or alternatively, the non-porous electrode support material can comprise a metal or a combination of a metal and its metal oxide, or a metal and its salt. The electrode support material is preferably formed of a material sufficiently chemically inert for the operation of the sensor. An electrode that is dedicated as a reference electrode can comprise a porous material and/or a non-porous material but without a catalyst.

Each electrode 510 is electrically coupled to a respective electrical connection 130 to provides an electrical connection between the electrodes 510 and the switching circuit 110. For example, the first electrode 511 is electrically coupled to the first electrical connection 131, the second electrode 512 is electrically coupled to the second electrical connection 132, and the optional third electrode 513 is electrically coupled to the third electrical connection 133. The electrical connections 130 can pass through one or more channels 530 defined in the housing 500 such that the electrical connections extend from the electrodes 510 to the switching circuit 110 (e.g., to the respective EC-side terminals 141-143 in the switching circuit 110). The channels 530 include fluid-tight seals to prevent gas from passing therethrough.

The electrolyte 520 is in contact with (e.g., direct physical contact with) the electrodes 510 and is disposed in the housing 500. The electrolyte 520 includes an electrolytic or ionic material that can transport charge between the working electrode and the counter electrode (e.g., between the first and second electrodes 511, 512 when the switching circuit 110 is in the first state). For example, the electrolyte 520 can comprise an ionic material such as an acid, base, a zwitterionic material, and/or a salt. The electrolyte 520 can be solid, can be a viscous liquid, such as a gel, or can be a polymer (e.g., a rigid and porous polymer) such as polybenzimidazole (PBI), styrenic block copolymers (TPS), a perfluorosulfonic acid polymer (e.g., Nafion® available from The Chemours Company) infused with an organic or inorganic acid. Examples of an inorganic acid include sulfuric acid and phosphoric acid. The electrolyte 520 can be sufficiently non-porous to prevent gasses from passing therethrough.

Additional details regarding the electrochemical cell 100 and/or other aspects of this disclosure are described in U.S. Patent Application Publication No. 2017/0336343 (“the '343 Publication”), titled “Integrated sensing device for detecting gasses,” published on Nov. 23, 2017, which is hereby incorporated by reference.

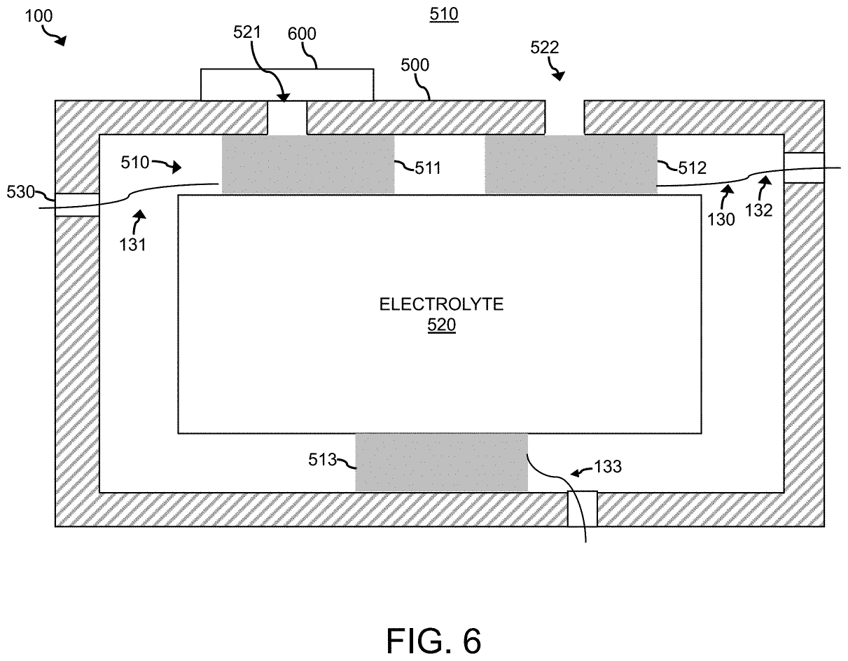

FIG. 6 is a block diagram of the electrochemical cell 100 illustrated in FIGS. 1-4 according to another embodiment. In this embodiment, a gas-selective filter 600 is disposed on the first vent hole 521. In an alternative embodiment, the gas-selective filter 600 can be disposed in the first vent hole 521. The gas-selective filter 600 can prevent one or more gases in the ambient environment from passing through the gas-selective filter 600 and into the first vent hole 521 to contact the first electrode 511. In some embodiments, the gas-selective filter 600 only allows one or more gases (or gas types) in the environment 510 to pass through the gas-selective filter 600 and into the first vent hole 521 to contact the first electrode 511.

In one example, the first and second electrodes 511, 512 are identical or substantially identical and comprise a catalyst that is sensitive to multiple gases. The gas-selective filter 600 can be configured to prevent a first gas or first gases from passing into the first vent hole 521 and contacting the first electrode 511. The concentration of the first gas or first gases can be determined by placing the switching circuit 110 in the first state to measure the concentration and/or composition of the filtered multiple gases (i.e., without the first gas(es)) and placing the switching circuit 110 in the second state to measure the concentration and/or composition of the unfiltered multiple gases. The concentration of the first gas(es) is the difference between these two concentrations (e.g., the concentration of (a) total unfiltered multiple gases in the environment 510—(b) filtered multiple gases). The switching circuit 110 can be placed in the first state before or after being placed in the second state. Measuring the concentration and/or composition of the gases in the first and second states can include measuring the impedance between a pair of electrodes in the electrochemical cell 100, for example between the electrode that functions as the working electrode and the electrode that functions as the counter electrode.

In a specific example, the first and second electrodes 511, 512 can comprise or consist of a catalyst, such as platinum, that is sensitive to a broad range of organic and inorganic gasses. The gas-selective filter 600 comprises or consists of a material, such as activated carbon that precludes organic gasses (e.g., volatile organic compounds) from passing through, while allowing inorganic gasses to pass through. The concentration of organic gases can be determined by placing the switching circuit 110 in the first state to measure the concentration and/or composition of inorganic gases and placing the switching circuit 110 in the second state to measure the concentration and/or composition of the organic and inorganic gases. The concentration of the organic gases is the difference between these two concentrations (e.g., the concentration of (a) organic+inorganic gases in the environment 510—(b) inorganic gases). The switching circuit 110 can be placed in the first state before or after being placed in the second state. Thus, the electrochemical gas sensor apparatus 10 can directly measure the concentration of inorganic gases in the environment 510 and can indirectly measure the concentration of organic gases in the environment 510 even though a gas-selective filter may not be available (or may be expensive) to filter out the inorganic gases so that the concentration of organic gases in the environment can be measured directly.

Measuring the concentration and/or composition of the gases in the first and second states can include measuring the impedance between a pair of electrodes in the electrochemical cell 100, for example between the electrode that functions as the working electrode and the electrode that functions as the counter electrode.

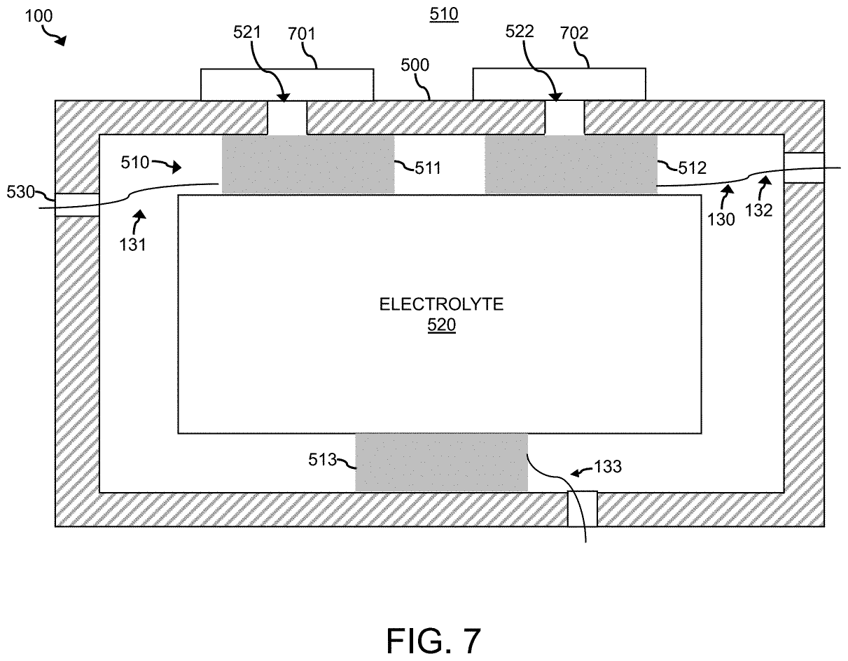

FIG. 7 is a block diagram of the electrochemical cell 100 illustrated in FIGS. 1-4 according to another embodiment. In this embodiment, a first gas-selective filter 701 is disposed on the first vent hole 521 and a second gas-selective filter 702 is disposed on the second vent hole 522. In an alternative embodiment, the first gas-selective filter 701 and/or the second gas-selective filter 702 can be disposed in the first vent hole 521 and/or in the second vent hole 522, respectively. The first gas-selective filter 701 can be the same as or different gas-selective filter 702.

The first gas-selective filter 701, the second gas-selective filter 702, and/or the gas-selective filter 600 can include or can consist of a zeolite. The zeolite can filter by molecular size to only allow gas molecules that are smaller than or equal to a predetermined maximum molecular size to pass through and into the corresponding vent hole. Additionally or alternatively, the zeolite can absorb a gas or gas species (e.g., ethanol). Additionally or alternatively, the zeolite can function as a catalyst to breakdown unwanted gas molecules. The first gas-selective filter 701, the second gas-selective filter 702, and/or the gas-selective filter 600 can alternatively include or consist of an absorptive material such as an activated carbon, or a catalytic material, for example as disclosed in U.S. Provisional Application No. 63/143,757, titled “Compact, microcomposite gas filter,” filed on Jan. 29, 2021, which is hereby incorporated by reference.

In one embodiment, the first and second gas-selective filters 701, 702 are different such that they filter different gases. For example, the first gas-selective filter 701 can be configured to allow only a first gas or first gases to pass into the first vent hole 521 (e.g., to contact the first electrode 511) and the second gas-selective filter 702 can be configured to allow only a second gas or second gases to pass into the second vent hole 522 (e.g., to contact the second electrode 512). This configuration is preferably used when the first and second electrodes 511, 512 comprise or consist of the same catalysts (e.g., when the first and second electrodes 511, 512 are identical or substantially identical).

For example, the first gas-selective filter 701 can be configured to allow only gases A and B to pass into the first vent hole 521, and the second gas-selective filter 702 can be configured to allow only gas A to pass into the second vent hole 522. When the switching circuit 110 is in the first state, the electrochemical gas sensor apparatus 10 can measure the total concentration of gases A and B in the environment 510. When the switching circuit 110 is in the second state, the electrochemical gas sensor apparatus 10 can measure the concentration of gas B in the environment 510. The electrochemical gas sensor apparatus 10 can then determine the concentration of gas A in the environment 510 as the difference between (a) the total concentration of gases A and B in the environment 510 and (b) the concentration of gas B in the environment 510.

Measuring the concentration and/or composition of the gases in the first and second states can include measuring the impedance between a pair of electrodes in the electrochemical cell 100, for example between the electrode that functions as the working electrode and the electrode that functions as the counter electrode.

In a specific example, the first gas-selective filter 701 can be configured to allow only carbon monoxide (CO) and nitrogen dioxide (NO₂) to pass into the first vent hole 521, and the second gas-selective filter 702 can be configured to allow only CO to pass into the second vent hole 522. For example, the first gas-selective filter 701 can comprise a zeolite with size selective less than or equal to 5 Å to selectively allow CO and NO₂ to pass into the first vent hole. Different zeolite structures such as ZSM and FAU can be used to achieve this. The second gas-selective filter 701 can comprise a zeolite with size selective less than or equal to 3 Å to selectively allow CO to pass into the second vent hole. When the switching circuit 110 is in the first state, the electrochemical gas sensor apparatus 10 can measure the total concentration of CO and NO₂ in the environment 510. When the switching circuit 110 is in the second state, the electrochemical gas sensor apparatus 10 can measure the concentration of CO in the environment 510. The electrochemical gas sensor apparatus 10 can then determine the concentration of gas NO₂ in the environment 510 as the difference between (a) the total concentration of CO and NO₂ in the environment 510 and (b) the concentration of CO in the environment 510.

In another embodiment, the first and second gas-selective filters 701, 702 are the same or substantially the same such that they filter the same gases. For example, the first and second gas-selective filters 701, 702 can be configured to allow only a first gas or first gases to pass into the first and second vent hole 521, 522, respectively (e.g., to contact the first and second electrodes 511, 512, respectively). This configuration is preferably used when the first and second electrodes 511, 512 comprise or consist of different catalysts (e.g., when the first and second electrodes 511, 512 are different). For example, the first and second gas-selective filters 701, 702 can comprise or can consists of a zeolite such as ZSMS, with first and second electrodes 511, 512 comprising Pt and Au catalysts, respectively, to differentiate between CO and HCHO.

In some embodiments, the electrochemical cell 100 can include a first array of electrodes 510 that are identical or substantially identical to one another and a second array of electrodes 510 that are different than each other. Gas-selective filters can be placed over or in the vent holes for any of the electrodes as desired.

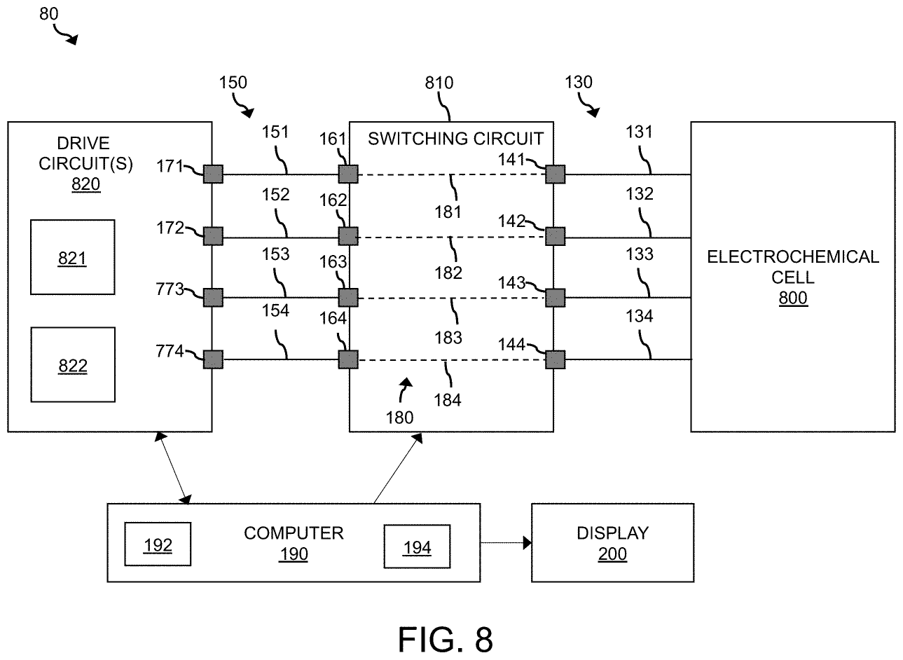

FIG. 8 is a block diagram of an electrochemical gas sensor apparatus 80 according to another embodiment. The apparatus 80 includes an electrochemical cell 800, a switching circuit 810, and one or more drive circuits 820. The electrochemical cell 800, switching circuit 810, and drive circuit(s) 820 are the same as electrochemical cell 100, switching circuit 110, and drive circuit 120, respectively, except as described herein. The apparatus 80 can also include a computer 190 and/or a display 200, which can be the same as discussed above.

In one embodiment, the electrochemical cell 800 is the same as electrochemical cell 100. In one embodiment, the electrochemical cell 800 include at least four electrodes. Each electrode is electrically coupled to a respective electrical connection 130 (e.g., electrical connections 131-134). For example, a first electrical connection 131 can electrically couple the switching circuit 810 (e.g., the first EC-side terminal 141 in the switching circuit 810) to a first electrode in the electrochemical cell 800, a second electrical connection 132 can electrically couple the switching circuit 810 (e.g., a second EC-side terminal 142 in the switching circuit 810) to a second electrode in the electrochemical cell 800, a third electrical connection 133 can electrically couple the switching circuit 810 (e.g., a third EC-side terminal 143 in the switching circuit 810) to a third electrode in the electrochemical cell 800, and an optional fourth electrical connection 134 can electrically couple the switching circuit 810 (e.g., an optional fourth EC-side terminal 144 in the switching circuit 810) to an optional fourth electrode in the electrochemical cell 800. In a preferred embodiment, the third and fourth electrodes can function as first and second reference electrodes, respectively.

The switching circuit 810 is electrically coupled to the drive circuit(s) 820 using a plurality of electrical connections 150. For example, a first electrical connection 151 can electrically couple the switching circuit 810 (e.g., a first DC-side terminal 161 in the switching circuit 810) to a working electrode-terminal 170 in the drive circuit(s) 820. A second electrical connection 152 can electrically couple the switching circuit 810 (e.g., a second DC-side terminal 162 in the switching circuit 810) to a counter-electrode terminal 171 in the drive circuit(s) 820. A third electrical connection 153 can electrically couple the switching circuit 810 (e.g., a third DC-side terminal 163 in the switching circuit 810) to a first reference-electrode terminal 773 in the drive circuit(s) 820. An optional fourth electrical connection 154 can electrically couple the switching circuit 810 (e.g., an optional fourth DC-side terminal 164 in the switching circuit 810) to an optional second reference-electrode terminal 774 in the drive circuit(s) 820.

FIG. 8 illustrates the first state of the switching circuit 810 in which the first EC-side terminal 141 is electrically coupled to the first DC-side terminal 161, the second EC-side terminal 142 is electrically coupled to the second DC-side terminal 162, the third EC-side terminal 143 is electrically coupled to the third DC-side terminal 163, and the optional fourth EC-side terminal 144 is electrically coupled to the optional fourth DC-side terminal 164. Thus, in the first state the first electrode in the electrochemical cell 800 is electrically coupled to the working-electrode terminal 171 in the drive circuit(s) 820, the second electrode in the electrochemical cell 800 is electrically coupled to the counter-electrode terminal 172 in the drive circuit(s) 820, the third electrode in the electrochemical cell 800 is electrically coupled to the first reference-electrode terminal 773 in the drive circuit(s) 820, and the optional fourth electrode in the electrochemical cell 800 is electrically coupled to the optional second reference-electrode terminal 774 in the drive circuit(s) 820.

The EC-side terminals 141-144 and the DC-side terminals 161-164 can be electrically coupled using switching electrical connections 181-184 (in general, switching connections 180), respectively. The switching circuit 810 can include additional or fewer switching electrical connections 180 in other embodiments. The switching circuit 810 can comprise a circuit board, a solid-state circuit, an integrated circuit, or another circuit. The switching circuit 810 can include a plurality of physical switches that alter the electrical path between the EC-side terminals 141-144 and the DC-side terminals 161-164.

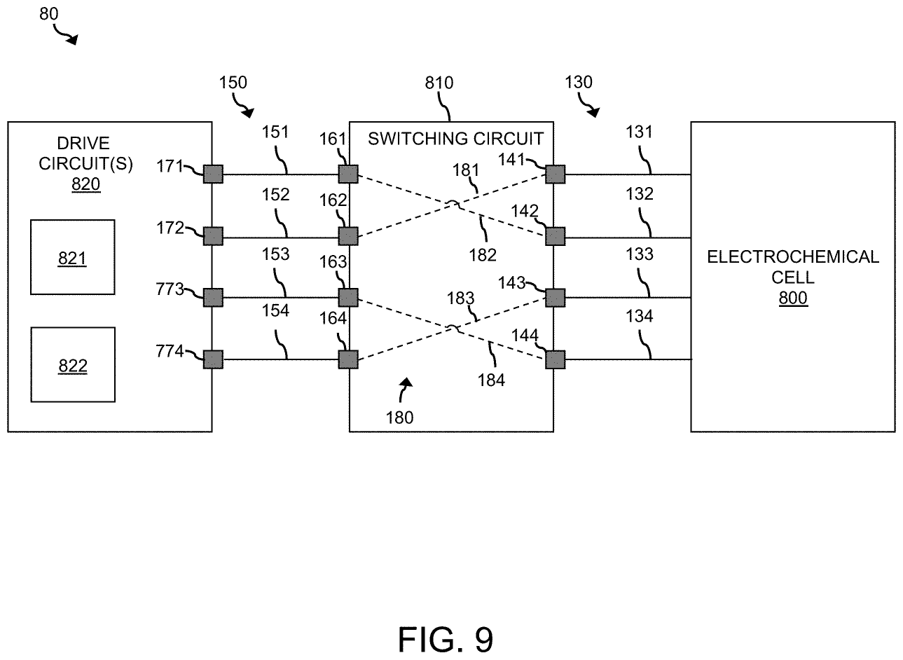

The switching circuit 810 can have multiple states, similar to switching circuit 110. For example, in another state switching electrical connection 182 can electrically couple EC-side terminal 142 and DC-side terminal 161 and switching electrical connection 181 can electrically couple EC-side terminal 141 and DC-side terminal 162, for example as illustrated in FIG. 9. Additionally or alternatively, in another state switching electrical connection 184 can electrically couple EC-side terminal 144 and DC-side terminal 163, and switching electrical connection 183 can electrically couple EC-side terminal 143 and DC-side terminal 164, for example as illustrated in FIG. 9. In another embodiment, EC-side terminal 144 can be electrically coupled to DC-side terminal 161 and EC-side terminal 144 can be electrically coupled to DC-side terminal 162. In another embodiment, EC-side terminal 143 can be electrically coupled to DC-side terminal 162 and EC-side terminal 143 can be electrically coupled to DC-side terminal 161. In general, any EC-side terminal can be electrically coupled to any DC-side terminal. Multiple EC-side terminals are preferably not electrically coupled to the same DC-side terminal, and multiple DC-side terminals are preferably not electrically coupled to the same EC-side terminal.

The drive circuit(s) 820 includes circuitry for driving the electrodes in the electrochemical cell 800 and for sensing a change in resistance or impedance of the electrodes, which can indicate the presence or concentration of one or more gases. The drive circuit(s) 820 can comprise one or more galvanostats or one or more potentiostats in some embodiments. The drive circuit(s) 820 can also comprise a voltmeter, a frequency analyzer, a function generator, an oscilloscope, and/or a network analyzer.

The drive circuit(s) 820 can include a first drive circuit 821 and a second drive circuit 822. Each drive circuit 821, 822 is electrically coupled to at least two terminals 171, 172, 773, 774. Each drive circuit 821, 822 can comprise one or more galvanostats or one or more potentiostats. In addition, each derive circuit 821, 822 can comprise a voltmeter, a frequency analyzer, a function generator, an oscilloscope, and/or a network analyzer.

In one embodiment, the drive circuits 821, 822 are electrically coupled to the same terminals. For example, the drive circuits 821, 822 can be electrically coupled to working-electrode terminal 171 and counter-electrode terminal 172 and can be optionally electrically coupled to first reference-electrode terminal 773 or second reference-electrode terminal 774. The first drive circuit 821 can operate at a first frequency and the second drive circuit 822 can operate at a second frequency that is different than the first frequency. The first and second drive circuits 821, 822 can drive the electrochemical cell 800 simultaneously or separately (e.g., sequentially). When the first and second drive circuits 821, 822 can drive the electrochemical cell 800 separately (e.g., sequentially), there can be a brief or momentary overlap when one or more of the connections (e.g., terminal connection to a respective electrode(s)) of each of the first and second drive circuits 821, 822 are simultaneously connected.

The first frequency can be relatively high compared to the second frequency, and the second frequency can be relatively low compared to the first frequency. The first frequency can correspond to a first mode for driving the electrochemical cell 800, such as high-frequency operation (e.g., for making “AC” measurements of the electrochemical cell 800 which can be the same as or similar to those performed in electrochemical impedance spectroscopy and/or voltammetry). The first frequency can comprise a first frequency range such that the AC measurements are made over the first frequency range. The first drive circuit 821 can iteratively or stepwise drive the electrochemical cell 800 over the first frequency range in a loop to provide the AC measurements over the first frequency range of about 1 kHz to about 1 MHz, including about 250 kHz, about 500 kHz, about 750 kHz, and any value or range between any two of the foregoing frequencies.

The second frequency can correspond to a second mode for driving the electrochemical cell 800, such as low-frequency and/or low-noise operation measurements of the electrochemical cell 800). The second frequency can comprise a second frequency range such that the low-frequency or zero-frequency measurements are made over the second frequency range of about 0 Hz to about 1 kHz, including about 250 Hz, about 500 Hz, about 750 Hz, and any value or range between any two of the foregoing frequencies.

In another embodiment, the drive circuits 821, 822 are electrically coupled to different terminal groups. For example, the first drive circuit 821 can be electrically coupled to a first terminal group, such as working-electrode terminal 171, counter-electrode terminal 172 and first reference-electrode terminal 773 while the second drive circuit 821 can be electrically coupled to a second terminal group, such as working-electrode terminal 171, counter-electrode terminal 172 and second reference-electrode terminal 774.

In one embodiment, the first and second drive circuits 821, 822 are electrically coupled to the same reference electrode (e.g., to the same reference electrode terminal) but to different working and counter electrodes (e.g., to different working-electrode terminals and counter-electrode terminals, respectively). The first and second drive circuits 821, 822 are preferably driven separately or sequentially in this configuration to electrically isolate the first and second drive circuits 821, 822 and the corresponding electrodes.

In another embodiment, the first and second drive circuits 821, 822 are electrically coupled to different reference electrodes (e.g., to different reference electrode terminals) but to the same working and counter electrodes (e.g., to the same working-electrode terminals and the same counter-electrode terminals). The first and second drive circuits 821, 822 are preferably driven separately or sequentially in this configuration to electrically isolate the first and second drive circuits 821, 822 and the corresponding electrodes.

In another embodiment, the first and second drive circuits 821, 822 are electrically coupled to different reference electrodes (e.g., to different reference electrode terminals) and to different working and counter electrodes (e.g., to different working-electrode terminals and to different counter-electrode terminals, respectively). The first and second drive circuits 821, 822 can be driven simultaneously in this embodiment since the first and second drive circuits 821, 822 are electrically isolated from each other. Alternatively, the first and second drive circuits 821, 822 can be driven separately or sequentially.

There can be additional or fewer electrode terminals in other embodiment. For example, the first drive circuit 821 can be electrically coupled to a first drive-electrode terminal, a first counter-electrode terminal, and an optional first reference-electrode terminal. In addition, the second drive circuit 821 can be electrically coupled to a second drive-electrode terminal, a second counter-electrode terminal, and an optional second reference-electrode terminal. The first and second drive-electrode terminals can be electrically coupled to the same or different DC-side terminal(s) (e.g., DC-side terminal 161, 162, 163, 164, and/or another DC-side terminal). The first and second counter-electrode terminals can be electrically coupled to the same or different DC-side terminal(s) (e.g., DC-side terminal 161, 162, 163, 164, and/or another DC-side terminal). The first and second reference-electrode terminals can be electrically coupled to the same or different DC-side terminal(s) (e.g., DC-side terminal 161, 162, 163, 164, and/or another DC-side terminal).

The drive circuits 821, 822 and/or the switching circuit 810 can be in electrical communication with the computer 190. The microprocessor 192 can be configured to receive electrical signals from each drive circuit 821, 822 that correspond to the current and/or resistance of the electrochemical cell 800 (e.g., of the electrodes that are electrically coupled to the respective drive circuit 821, 822) as a function of time. A change in current and/or resistance can correspond to the presence of one or more detected gases and the magnitude in the change in current and/or resistance can correspond to the concentration of the detected gas(es) in the environment. The memory 194 can include a database, lookup tables, and/or mathematical models to determine the concentration of the detected gas(es) as a function of the change in current and/or resistance. The microprocessor 192 can be configured to determine the concentration of the detected gas(es) using the database, lookup table, and/or mathematical model in the memory 194. In addition, the microprocessor can be configured to generate an output signal that causes the display 200 to graphically display the concentration of the detected gas(es).

In some embodiments, the microprocessor 192 can be configured to send a control signal to each drive circuit 821, 822 to set the respective frequency of each drive circuit 821, 822. For example, the microprocessor 192 can be configured to generate a first control signal that corresponds to the first frequency of the first drive circuit 821 and/or a second control signal that corresponds to the second frequency of the second drive circuit 822. In some embodiments, the microprocessor 192 can send the control signals to either or both drive circuits 821, 822 in response to a user input signal, which the computer 190 can receive via a wired or a wireless connection.

In another example, the microprocessor 192 can be configured to send a control signal to the switching circuit 810 that causes the switching circuit 810 to be configured in a desired state. For example, the control signal can cause the switching circuit 810 to transition to the first state (e.g., as illustrated in FIG. 8), the second state (e.g., as illustrated in FIG. 9), or another state. The control signal can cause one or more switches (e.g., physical switches, solid-state switches, or other switches) to change state to alter the switching connections 180 between the EC-side terminals 141-143 and the DC-side terminals 161-163. In some embodiments, the microprocessor 192 can send the control signal to the switching circuit 810 in response to a user input signal, which the computer 190 can receive via a wired or a wireless connection.

Two or more components of the electrochemical gas sensor apparatus 80 can be disposed or combined in a single housing. For example, the switching circuit 810 and the drive circuit 820 can be disposed or combined in a single housing. Additionally or alternatively, the computer 190 and the drive circuit(s) 820 can be disposed or combined in a single housing. In another example, the electrochemical cell 800, the switching circuit 810, and the drive circuit(s) 820 can be combined or disposed in a single housing. In yet another example, the electrochemical cell 800, the switching circuit 810, the drive circuit(s) 820, the computer 190, and/or the display 200 can be combined or disposed in a single housing.

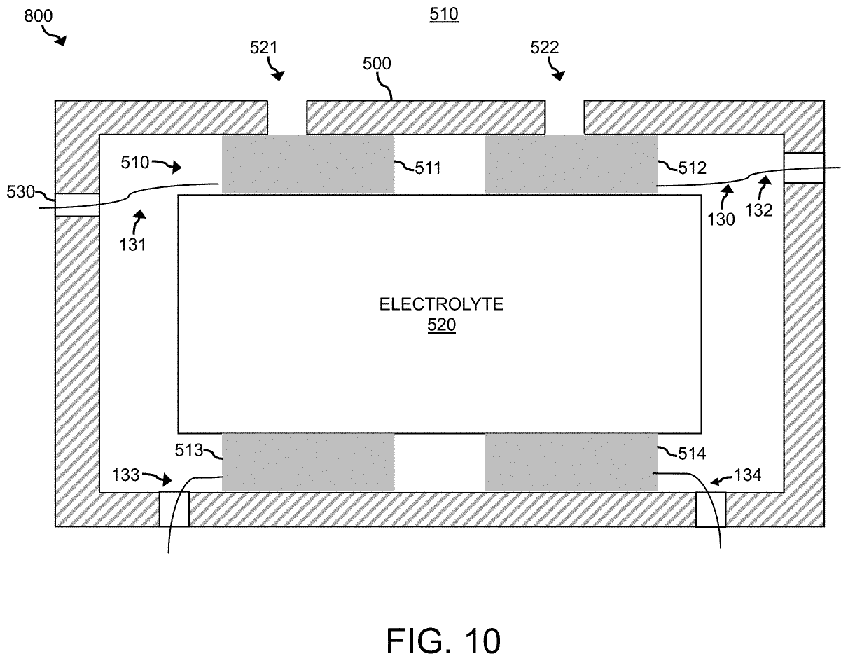

FIG. 10 is a block diagram of the electrochemical cell 800 illustrated in FIGS. 8 and 9 according to an embodiment. The electrochemical cell 800 is the same as electrochemical cell 100 except that electrochemical cell 800 includes an optional fourth electrode 514 disposed in the housing 500. In a preferred embodiment, the optional third electrode 513 functions as a first reference electrode and the optional fourth electrode 514 functions as a second reference electrode. The electrochemical cell 800 can include additional or fewer electrodes in other embodiments. In an embodiment, electrochemical cell 800 is the same as electrochemical cell 100. Additional details regarding example electrode configurations are described in U.S. Pat. No. 10,908,117, titled “Low Impedance Sensor for Low Density Materials,” and U.S. Patent Application Publication No. 2019/0227024, titled “Chip-Scale Sensing Device for Low Density Material,” which are hereby incorporated by reference.

In an embodiment, the optional third and fourth electrodes 513, 514 are identical or substantially identical (e.g., within manufacturing tolerances). For example, the optional third and fourth electrodes 513, 514 can be configured and arranged such that the electrical impedance between the third electrode 513 and the first and/or second electrodes 511, 512 is the same or about the same as the electrical impedance between the fourth electrode 514 and the first and/or second electrodes 511, 512. In another embodiment, the optional third and fourth electrodes 513, 514 are different. For example, the optional third and fourth electrodes 513, 514 can be configured and arranged such that the electrical impedance between the third electrode 513 and the first and/or second electrodes 511, 512 is different than the electrical impedance between the fourth electrode 514 and the first and/or second electrodes 511, 512.

When the electrochemical cell 800 includes the third and fourth electrodes 513, 514 and when the switching circuit 810 is in the first state (e.g., as illustrated in FIG. 8), the first drive circuit 821 can be electrically coupled to the first electrode 511, the second electrode 512, and the third electrode 513 via the working-electrode terminal 171, counter-electrode terminal 172 and first reference-electrode terminal 773, respectively, such that first electrode 511 functions as the working electrode, the second electrode 512 functions as the counter electrode, and the third electrode 513 functions as the reference electrode. Further, in this configuration and when the switching circuit 810 is in the first state, the second drive circuit 822 can be electrically coupled to the first electrode 511, the second electrode 512, and the fourth electrode 514 via the working-electrode terminal 171, counter-electrode terminal 172 and second reference-electrode terminal 774, respectively, such that first electrode 511 functions as the working electrode, the second electrode 512 functions as the counter electrode, and the fourth electrode 514 functions as the reference electrode. When the switching circuit 810 is in the second state, the first electrode 511 can function as the counter electrode and the second electrode 512 can function as the working electrode in the first and second drive circuits 821, 822 where the respective third and fourth electrodes 513, 514 can continue to function as reference electrodes.

In another embodiment, the first drive circuit 821 and/or the second drive circuit 822 can be electrically coupled to both the third and fourth electrodes 513, 514. For example, when the switching circuit 810 is in the first state (e.g., as illustrated in FIG. 8), the first drive circuit 821 and/or the second drive circuit 822 can be electrically coupled to the first electrode 511, the second electrode 512, the third electrode 513, and the fourth electrode 514 via the working-electrode terminal 171, counter-electrode terminal 172, first reference-electrode terminal 773, and second reference terminal 774, respectively, such that first electrode 511 functions as the working electrode, the second electrode 512 functions as the counter electrode, and the third electrode 513 functions as a first reference electrode, and the fourth electrode 514 functions as a second reference electrode.

The electrochemical cell 800 is electrically coupled to the switching circuit 810 using a plurality of electrical connections 130. For example, the first electrical connection 131 can electrically couple the switching circuit 810 (e.g., the first EC-side terminal 141 in the switching circuit 810) to the first electrode 511 in the electrochemical cell 800, the second electrical connection 132 can electrically couple the switching circuit 810 (e.g., the second EC-side terminal 142 in the switching circuit 810) to the second electrode 512 in the electrochemical cell 800, the third electrical connection 133 can electrically couple the switching circuit 810 (e.g., the third EC-side terminal 143 in the switching circuit 810) to the third electrode 513 in the electrochemical cell 800, and the fourth electrical connection 134 can electrically couple the switching circuit 810 (e.g., the fourth EC-side terminal 144 in the switching circuit 810) to the fourth electrode 514 in the electrochemical cell 800.

The electrochemical cell 800 can include a first gas-selective filter (e.g., first gas-selective filter 701) disposed on or in the first vent hole 521 and/or a second gas-selective filter (e.g., second gas-selective filter 702) disposed on or in the second vent hole 522.

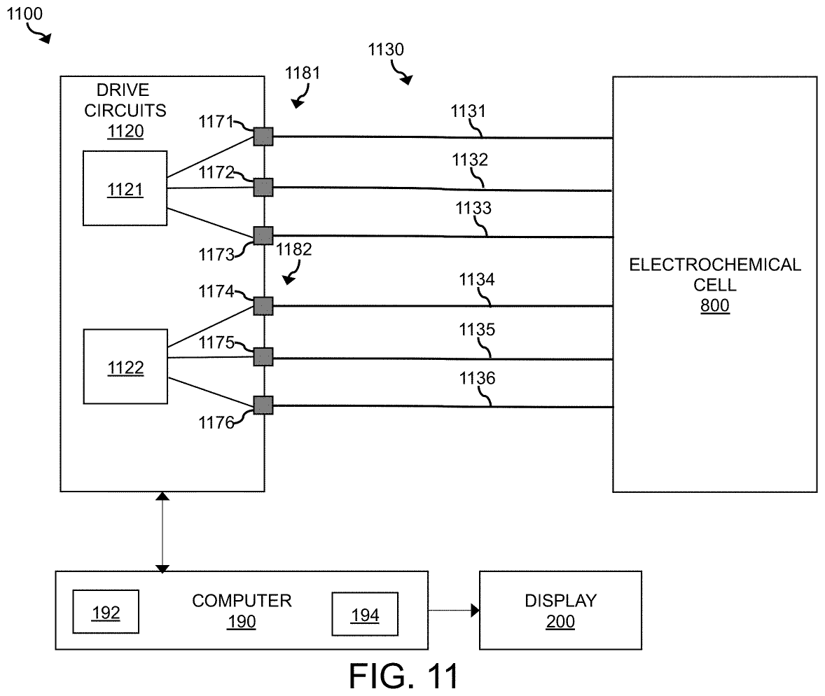

FIG. 11 is a block diagram of an electrochemical gas sensor apparatus 1100 according to another embodiment. The apparatus 1100 includes an electrochemical cell 800 that is electrically coupled to a plurality of drive circuits 1120. Drive circuits 1120 are the same as drive circuit(s) 820 except as discussed herein. Drive circuits 1120 include a first drive circuit 1121 and a second drive circuit 1122, which can be the same as the first drive circuit 821 and the second drive circuit 822, respectively. The first drive circuit 1121 is electrically coupled to a first group 1181 of electrode terminals. The first group 1181 of electrode terminals includes a first driving-electrode terminal 1171, a first counter-electrode terminal 1172, and an optional first reference-electrode terminal 1173. The second drive circuit 1122 is electrically coupled to a second group 1182 of electrode terminals. The second group 1182 of electrode terminals includes a second driving-electrode terminal 1174, a second counter-electrode terminal 1174, and an optional second reference-electrode terminal 1174.

Each electrode terminal is electrically coupled to an electrode in the electrochemical cell 800 via respective electrical connections 1130, which can include wires, electrical conduits, and/or other electrical connections. For example, the first driving-electrode terminal 1171 is electrically coupled to a first electrode in electrochemical cell 800 via a first electrical connection 1131. The first counter-electrode terminal 1172 is electrically coupled to a second electrode in electrochemical cell 800 via a second electrical connection 1132. The optional first reference-electrode terminal 1173 is electrically coupled to a third electrode in electrochemical cell 800 via an optional third electrical connection 1133. The second driving-electrode terminal 1174 is electrically coupled to a fourth electrode in electrochemical cell 800 via a fourth electrical connection 1134. The second counter-electrode terminal 1175 is electrically coupled to a fifth electrode in electrochemical cell 800 via a fifth electrical connection 1135. The optional second reference-electrode terminal 1176 is electrically coupled to a sixth electrode in electrochemical cell 800 via an optional sixth electrical connection 1136.

As discussed herein, the first and second drive circuits can be electrically coupled to the same or different groups of electrodes in the electrochemical cell. For example, when the first and second drive circuits 1121, 1122 are electrically coupled to the same group of electrodes (i.e., the first and second groups of electrodes are the same), the first and second driving-electrode terminals 1171, 1174 are electrically coupled to the same electrode (i.e., the first and fourth electrodes are the same) via the respective first and fourth electrical connections 1131, 1134. In addition, when the first and second drive circuits 1121, 1122 are electrically coupled to the same group of electrodes, the first and second counter-electrode terminals 1172, 1175 are electrically coupled to the same electrode (i.e., the second and fifth electrodes are the same) via the respective second and fifth electrical connections 1132, 1135. In addition, when the first and second drive circuits 1121, 1122 are electrically coupled to the same group of electrodes, the first and second reference-electrode terminals 1173, 1176 are electrically coupled to the same electrode (i.e., the third and sixth electrodes are the same) via the respective third and sixth electrical connections 1133, 1136. When the first and second drive circuits 1121, 1122 are electrically coupled to the same group of electrodes, the first and second drive circuits 1121, 1122 are preferably driven separately or sequentially in this configuration to electrically isolate the first and second drive circuits 1121, 1122 and the corresponding electrodes.

When the first and second drive circuits 1121, 1122 are electrically coupled to the different groups of electrodes (i.e., the first and second groups of electrodes are different), at least one of terminals 1171-1173 is electrically coupled to a different electrode than at least one of terminals 1174-1176, respectively. For example, the first and second driving-electrode terminals 1171, 1174 can be electrically coupled to the different electrodes (i.e., the first and fourth electrodes are different) via the respective first and fourth electrical connections 1131, 1134. Additionally or alternatively, the first and second counter-electrode terminals 1172, 1175 can be electrically coupled to the different electrodes (i.e., the second and fifth electrodes are different) via the respective second and fifth electrical connections 1132, 1135. Additionally or alternatively, the first and second reference-electrode terminals 1173, 1176 can be electrically coupled to the different electrodes (i.e., the third and sixth electrodes are different) via the respective third and sixth electrical connections 1133, 1136. When the first and second drive circuits 1121, 1122 are electrically coupled to at least one common electrode, the first and second drive circuits 1121, 1122 are preferably driven separately or sequentially to electrically isolate the first and second drive circuits 1121, 1122 and the corresponding electrode(s). When the first and second drive circuits 1121, 1122 are not coupled to a common electrode (i.e., there is no overlap between the first and second groups of electrodes), the first and second drive circuits 1121, 1122 can be driven simultaneously since the first and second drive circuits 1121, 1122 and the respective electrodes are electrically isolated from each other. Alternatively, the first and second drive circuits 1121, 1122 can be driven separately or sequentially when the first and second drive circuits 1121, 1122 are not coupled to a common electrode.

The first and second drive circuits 1121, 1122 can operate at different frequencies, including zero Hz, for example as discussed above with respect to drive circuits 821, 822.

In some embodiments, drive circuits 821, 822 can be electrically coupled to electrode terminals 1171-1176 in the same or similar manner as discussed herein with respect to drive circuits 1121, 1122, respectively.

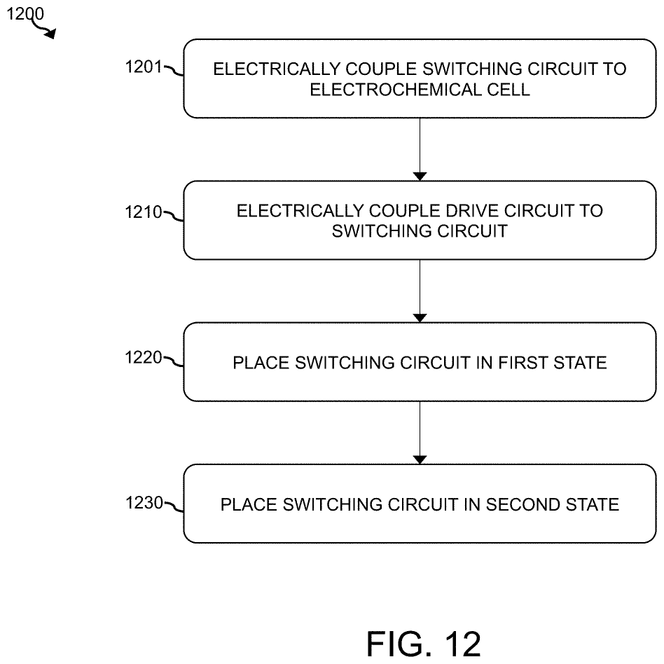

FIG. 12 is a flow chart of a method 1200 of operating an electrochemical gas sensor according to an embodiment. In step 1201, a switching circuit is electrically coupled to an electrochemical cell that includes a first electrode, a second electrode, and a third electrode. The electrochemical cell can include additional or fewer electrodes in other embodiments. In some embodiments, the electrochemical cell can be the same as or different than electrochemical cell 100 or electrochemical cell 800. The switching circuit can be the same as or different than switching circuit 110 or switching circuit 810.

In step 1210, a drive circuit is electrically coupled to the switching circuit. The drive circuit includes a working-electrode terminal, a counter-electrode terminal, and a reference-electrode terminal, or other appropriate sense, drive, and common terminals.

In step 1220, the switching circuit is placed in a first state in which the first electrode is electrically coupled to the working-electrode terminal, the second electrode is electrically coupled to the counter-electrode terminal, and the third electrode is electrically coupled to the reference-electrode terminal. In the first state, the first electrode functions as the working electrode, the second electrode functions as the counter electrode, and the third electrode functions as the reference electrode.

In step 1230, the switching circuit is placed in a second state in which the second electrode is electrically coupled to the working-electrode terminal, the first electrode is electrically coupled to the counter-electrode terminal, and the third electrode is electrically coupled to the reference-electrode terminal. In the second state, the second electrode functions as the working electrode, the first electrode functions as the counter electrode, and the third electrode functions as the reference electrode.

In the first state, a concentration of a first gas can be determined such as by using a processor in electrical communication with the drive circuit. In the second state, a concentration of a second gas can be determined such as by using the processor.

In some embodiments, the method 1200 can include filtering ambient gases with a first filter that only allows first and second gases to pass through to the first electrode. The method 1200 can also include filtering the ambient gases with a second filter that only allows the first gas to pass through to the second electrode. When the switching circuit is in the first state, the processor can be used to determine the total concentration of the first and second gases. When the switching circuit is in the second state, the processor can be used to determine the concentration of the first gas. The processor can determine the concentration of the second gas based on the total concentration of the first and second gases and the concentration of the first gas. For example, the concentration of the second gas can be equal to the difference between the total concentration of the first and second gases and the concentration of the first gas. In an embodiment, the first gas comprises or consists of NO₂ and the second gas comprises or consists of CO.

In another embodiment, the method 1200 can include filtering ambient gases with a first filter that only allows inorganic gases to pass through to the first electrode. When the switching circuit is in the first state, the processor can be used to determine a concentration of the inorganic gases. When the switching circuit is in the second state, the processor can be used to determine a total concentration of the organic and inorganic gases. The processor can determine the concentration of the organic gases based on the total concentration of the organic and inorganic gases and the concentration of the inorganic gases. For example, the concentration of the organic gases can be equal to the difference between total concentration of the organic and inorganic gases and the concentration of the inorganic gases.

In another embodiment, the method 1200 can include filtering ambient gases with a first filter that only allows organic gases to pass through to the first electrode. When the switching circuit is in the first state, the processor can be used to determine a concentration of the organic gases. When the switching circuit is in the second state, the processor can be used to determine a total concentration of the organic and inorganic gases. The processor can determine the concentration of the inorganic gases based on the total concentration of the organic and inorganic gases and the concentration of the organic gases. For example, the concentration of the inorganic gases can be equal to the difference between total concentration of the organic and inorganic gases and the concentration of the organic gases.

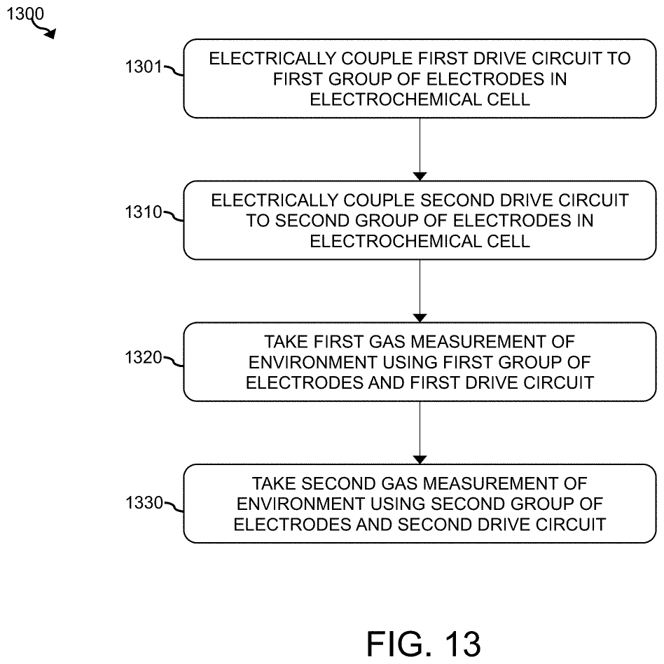

FIG. 13 is a flow chart of a method 1300 of sensing a gas according to an embodiment. In step 1301, a first drive circuit is electrically coupled to a first group of electrodes in an electrochemical cell. In step 1310, a second drive circuit is electrically coupled to a second group of electrodes in an electrochemical cell. One or more of the electrodes in the first group of electrodes can be the same as or different than one or more of the electrodes in the second group of electrodes.

In step 1320, the first drive circuit and the first group of electrodes are used to take a first gas measurement of an environment of the electrochemical cell. In step 1330, the second drive circuit and the second group of electrodes are used to take a second gas measurement of the environment.

In one embodiment, the first and second groups of electrodes can be the same. The first and second drive circuits can operate at different frequencies. For example, the first drive circuit can operate at a relatively high frequency to perform “AC” measurements of the first group of electrodes (e.g., which can be the same as or similar to those performed in electrochemical impedance spectroscopy and/or voltammetry). The first drive circuit can operate at a first frequency or a first frequency range such that the AC measurements are made at the first frequency or over the first frequency range. The first drive circuit can iteratively or stepwise drive the first group of electrodes over the first frequency range in a loop to provide the AC measurements over the first frequency range. The second drive circuit can operate at a relatively low frequency to perform low-frequency and/or low-noise measurements. The second frequency can comprise a second frequency range such that the measurements are made over the second frequency range. The first drive circuit can be electrically disconnected from the electrochemical cell (e.g., from the first group of electrodes) when the second drive circuit is electrically coupled to the electrochemical cell (e.g., to the second group of electrodes). Likewise, the second drive circuit can be electrically disconnected from the electrochemical cell (e.g., from the second group of electrodes) when the first drive circuit is electrically coupled to the electrochemical cell (e.g., to the first group of electrodes).

In another embodiment, the first and second groups of electrodes can be different. For example, the first group of electrodes can include a first working electrode that is sensitive to a first gas and the second group of electrodes can include a second working electrode that is sensitive to a second gas. The first gas measurement can include a measurement of the concentration of the first gas in the environment. The second gas measurement can include a measurement of the concentration of the second gas in the environment. When there is no common electrode in the first and second groups of electrodes (i.e., the first and second groups of electrodes comprise unique electrodes), the first and second drive circuits can be electrically coupled to the first and second groups of electrodes, respectively, simultaneously.

In another example, the first and second groups of electrodes include the same working and counter electrodes, but the first group of electrodes includes a first reference electrode and the second group of electrodes includes a second reference electrode. The first and second drive circuits can operate at different frequencies, including zero Hz, as discussed above.

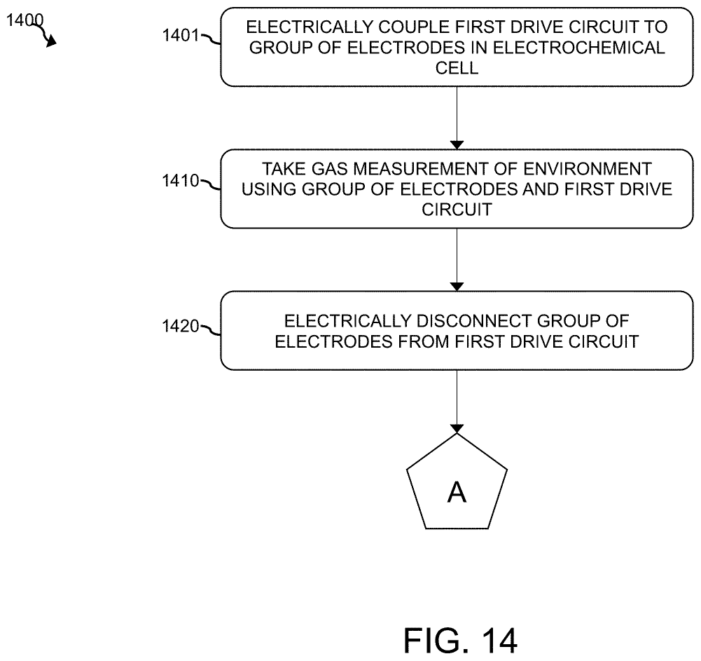

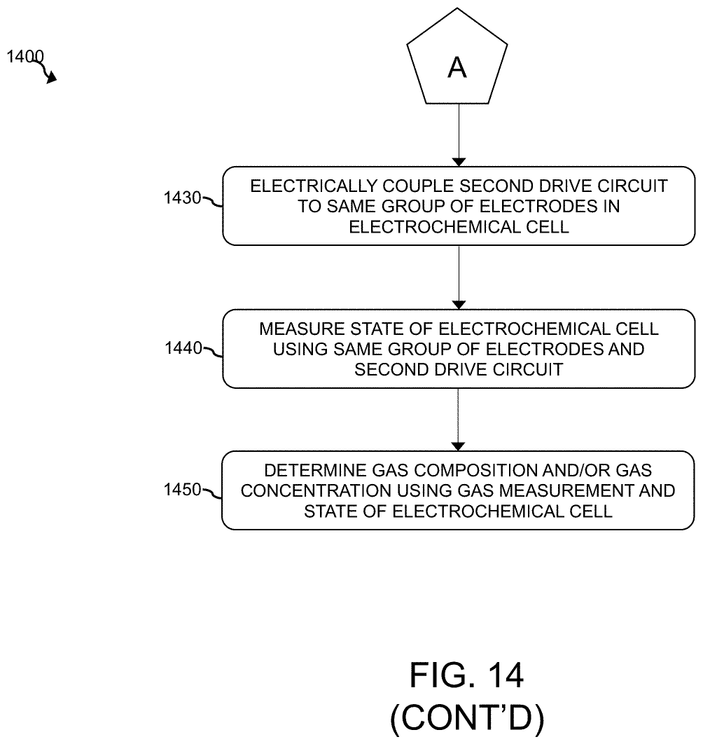

FIG. 14 is a flow chart of a method 1400 of sensing a gas according to an embodiment. In step 1401, a first drive circuit is electrically coupled to a group of electrodes in an electrochemical cell. A switching circuit can be used to electrically couple the first drive circuit to the group of electrodes.

In step 1410, the first drive circuit and the group of electrodes are used to take a gas measurement of an environment of the electrochemical cell. The gas measurement can comprise taking an electrical impedance measurement of the electrochemical cell, such as between the working and counter electrodes. In step 1420, the group of electrodes is electrically disconnected from the first drive circuit. The switching circuit can be used to electrically disconnect or decouple the first drive circuit from the group of electrodes.

In step 1430, a second drive circuit is electrically coupled to the same group of electrodes in the electrochemical cell as the first drive circuit. The switching circuit can be used to electrically couple the second drive circuit to the group of electrodes.

In step 1440, the second drive circuit and the group of electrodes are used to determine a state of the electrochemical cell. Steps 1430 and/or 1440 can occur after step 1420. Determining the state of the electrochemical cell can include determining the impedance of the electrochemical cell (e.g., electrochemical impedance spectroscopy). For example, the impedance between at least one pair of electrodes can be determined. In some embodiments, the impedance between each unique pair of electrodes can be determined. For example, in a 3-electrode system, the impedance (Zwe-re) between the working electrode and the reference electrode, the impedance (Zce-re) between the counter electrode and the reference electrode, and/or the impedance (Zwe-ce) between the working electrode and the counter electrode can be determined.

Additionally or alternatively, determining the state of the electrochemical cell can include determining one or more environmental conditions of the electrochemical cell. The environmental conditions can include temperature, relative humidity, atmospheric pressure, and/or conductivity at the electrode/electrolyte interface. Voltammetry or cyclic voltammetry (CV) can also be used to determine one or more metrics of the state of the electrochemical cell.

In step 1450, the gas measurement and the state of the electrochemical cell are used to determine a composition and/or a concentration of a gas in the environment. Additional details regarding this step and/or other aspects of this disclosure are described in the '141 patent, in U.S. Patent Application Publication No. 2020/0319137, titled “Electrochemical Gas Sensor With Varying Bias Voltage and Environmental Compensation,” published on Oct. 8, 2020, and/or in the '343 Publication, which are hereby incorporated by reference.

Electrochemical impedance spectroscopy can be used to improve the accuracy of the gas measurement. For example, the sensitivity of the cell to a gas may be a function in part of the electrochemical impedance (e.g., the impedance between a pair of electrodes in the electrochemical cell). In addition, the electrochemical cell's impedance may vary over time. By measuring the impedance of the electrochemical cell, it is possible to account for that fraction of the gas sensitivity of the electrochemical cell which is being impacted by the electrochemical cell's impedance—and include this variable into the calculation of the gas concentration being sensed.

The present invention should not be considered limited to the particular embodiments described above. Various modifications, equivalent processes, as well as numerous structures to which the present invention may be applicable, will be readily apparent to those skilled in the art to which the present invention is directed upon review of the present disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of the present application need not reside on a single computer or processor, but may be distributed in a modular fashion among a number of different computers or processors to implement various aspects of the present application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the present disclosure and claims include new and novel improvements to existing methods and technologies, which were not previously known nor implemented to achieve the useful results described above. Users of the present method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. An electrochemical gas sensor comprising:
an electrochemical cell comprising:
a first electrode;
a second electrode;
a third electrode; and
an electrolyte in contact with the first, second, and third electrodes;
a switching circuit electrically coupled to the electrochemical cell; and
a drive circuit electrically coupled to the switching circuit, the drive circuit having a working-electrode terminal, a counter-electrode terminal, and a reference-electrode terminal,
wherein:
the switching circuit has a first state in which the first electrode is electrically coupled to the working-electrode terminal, the second electrode is electrically coupled to the counter-electrode terminal, and the third electrode is electrically coupled to the reference-electrode terminal, and
the switching circuit has a second state in which the first electrode is electrically coupled to the counter-electrode terminal, the second electrode is electrically coupled to the working-electrode terminal, and the third electrode is electrically coupled to the reference-electrode terminal.

2. The sensor of claim 1, wherein the drive circuit comprises a galvanostat or a potentiostat.

3. The sensor of claim 2, wherein the drive circuit further comprises a voltmeter, a frequency analyzer, a function generator, an oscilloscope, and/or a network analyzer.

4. The sensor of claim 1, wherein:
the electrochemical cell comprises a housing, the first, second, and third electrodes disposed in the housing,
a first vent hole is defined in the housing to expose a portion of the first electrode, and
a second vent hole is defined in the housing to expose a portion of the second electrode.

5. The sensor of claim 4, further comprising a gas-selective filter disposed on or in the first vent hole.

6. The sensor of claim 5, wherein:
the gas-selective filter is configured to prevent first gases from passing through the gas-selective filter, and
the first and second electrodes comprise a same catalyst that is sensitive to multiple gases including the first gases.

7. The sensor of claim 6, wherein the first gases comprise organic gases.

8. The sensor of claim 5, wherein the gas-selective filter is a first gas-selective filter and the sensor further comprises a second gas-selective filter disposed on or in the second vent hole.

9. The sensor of claim 8, wherein the first and second gas-selective filters are configured to filter different gasses.

10. The sensor of claim 9, wherein:
the first gas-selective filter is configured to only allow carbon monoxide and nitrogen dioxide to pass through the first gas-selective filter,
the second gas-selective filter is configured to only allow carbon monoxide to pass through the second gas-selective filter,
when the switching circuit is in the first state, the sensor measures a total concentration of carbon monoxide and nitrogen dioxide in an environment of the sensor, and
when the switching circuit is in the second state, the sensor measures a concentration of carbon monoxide in the environment.

11. The sensor of claim 9, wherein the first and second electrodes comprise different catalysts.

12. The sensor of claim 4, wherein the first and second electrodes comprise different catalysts.

13. The sensor of claim 12, further comprising a gas-selective filter disposed on or in the first vent hole.

14. The sensor of claim 1, wherein:
the drive circuit is a first drive circuit, and
the working-electrode terminal, the counter-electrode terminal, and the reference-electrode terminal are a first working-electrode terminal, a first counter-electrode terminal, and a first reference-electrode terminal, respectively, and
the sensor further comprises a second drive circuit electrically coupled to the switching circuit, the second drive circuit having a second working-electrode terminal, a second counter-electrode terminal, and a second reference-electrode terminal,
wherein:
the switching circuit has a third state in which the first electrode is electrically coupled to the second working-electrode terminal, the second electrode is electrically coupled to the second counter-electrode terminal, and the third electrode is electrically coupled to the second reference-electrode terminal.

15. The sensor of claim 14, wherein:
when the switching circuit is in the first state, the second drive circuit is electrically decoupled from the electrochemical cell,
when the switching circuit is in the second state, the second drive circuit is electrically decoupled from the electrochemical cell, and
when the switching circuit is in the third state, the first drive circuit is electrically decoupled from the electrochemical cell.

16. An electrochemical gas sensor comprising:
an electrochemical cell comprising:
a first electrode;
a second electrode;
a third electrode;
a fourth electrode; and
an electrolyte in contact with the first, second, third, and fourth electrodes;
a switching circuit electrically coupled to the electrochemical cell; and
a drive circuit electrically coupled to the switching circuit, the drive circuit having a working-electrode terminal, a counter-electrode terminal, and a reference-electrode terminal,
wherein:
the switching circuit has a first state in which the first electrode is electrically coupled to the working-electrode terminal, the second electrode is electrically coupled to the counter-electrode terminal, and the third electrode is electrically coupled to the reference-electrode terminal, and
the switching circuit has a second state in which the first electrode is electrically coupled to the working-electrode terminal, the second electrode is electrically coupled to the counter-electrode terminal, and the fourth electrode is electrically coupled to the reference-electrode terminal.

17. The sensor of claim 16, wherein:
the electrochemical cell comprises a housing, the first, second, third, and fourth electrodes disposed in the housing,
a first vent hole is defined in the housing to expose a portion of the first electrode, and
a second vent hole is defined in the housing to expose a portion of the second electrode.

18. The sensor of claim 17, wherein the switching circuit has a third state in which the second electrode is electrically coupled to the working-electrode terminal, the first electrode is electrically coupled to the counter-electrode terminal, and the third electrode is electrically coupled to the reference-electrode terminal.

19. The sensor of claim 18, wherein the first and second electrodes comprise different catalysts.

20. The sensor of claim 19, further comprising a gas-selective filter disposed on or in the first vent hole.

21. The sensor of claim 20, wherein:
the gas-selective filter is configured to prevent first gases from passing through the gas-selective filter, and
the first and second electrodes comprise a same catalyst that is sensitive to multiple gases including the first gases.

22. The sensor of claim 20, wherein the gas-selective filter is a first gas-selective filter and the sensor further comprises a second gas-selective filter disposed on or in the second vent hole.

23. The sensor of claim 21, wherein the first and second gas-selective filters are configured to filter different gasses.

24. The sensor of claim 23, wherein:
the first gas-selective filter is configured to only allow carbon monoxide and nitrogen dioxide to pass through the first gas-selective filter,
the second gas-selective filter is configured to only allow carbon monoxide to pass through the second gas-selective filter,
when the switching circuit is in the first state, the sensor measures a total concentration of carbon monoxide and nitrogen dioxide in an environment of the sensor, and
when the switching circuit is in the second state, the sensor measures a concentration of carbon monoxide in the environment.

25. The sensor of claim 18, wherein the switching circuit has a fourth state in which the second electrode is electrically coupled to the working-electrode terminal, the first electrode is electrically coupled to the counter-electrode terminal, and the fourth electrode is electrically coupled to the reference-electrode terminal.

26. The sensor of claim 16, wherein the drive circuit comprises a galvanostat or a potentiostat.

27. The sensor of claim 26, wherein the drive circuit further comprises a voltmeter, a frequency analyzer, a function generator, an oscilloscope, and/or a network analyzer.

28. An electrochemical gas sensor comprising:
an electrochemical cell comprising:
a first electrode;
a second electrode;
a first reference electrode;
a second reference electrode; and
an electrolyte in contact with the first, second, first reference, and second reference electrodes;
a first drive circuit electrically coupled to the first electrode, the second electrode, and the first reference electrode; and
a second drive circuit electrically coupled to the first electrode, the second electrode, and the second reference electrode.

29. The sensor of claim 28, wherein the first drive circuit and the second drive circuit are configured to operate at different frequencies.

30. The sensor of claim 28, wherein an impedance between the first reference electrode and the first electrode is different than an impedance between the second reference electrode and the first electrode.

31. The sensor of claim 30, wherein an impedance between the first reference electrode and the second electrode is different than an impedance between the second reference electrode and the second electrode.

32. The sensor of claim 28, further comprising a switching circuit electrically coupled to the electrochemical cell, the first drive circuit, and the second drive circuit, wherein:
the first drive circuit includes a first working-electrode terminal, a first counter-electrode terminal, and a first reference-electrode terminal,
the second drive circuit includes a second working-electrode terminal, a second counter-electrode terminal, and a second reference-electrode terminal,
the switching circuit has a first state in which the first electrode is electrically coupled to the first working-electrode terminal, the second electrode is electrically coupled to the first counter-electrode terminal, and the first reference electrode is electrically coupled to the first reference-electrode terminal, and
the switching circuit has a second state in which the first electrode is electrically coupled to the first counter-electrode terminal, the second electrode is electrically coupled to the first working-electrode terminal, and the first reference electrode is electrically coupled to the first reference-electrode terminal.

33. The sensor of claim 32, wherein:
the switching circuit has a third state in which the first electrode is electrically coupled to the second working-electrode terminal, the second electrode is electrically coupled to the second counter-electrode terminal, and the second reference electrode is electrically coupled to the second reference-electrode terminal, and
the switching circuit has a fourth state in which the first electrode is electrically coupled to the second counter-electrode terminal, the second electrode is electrically coupled to the second working-electrode terminal, and the second reference electrode is electrically coupled to the second reference-electrode terminal.

34. A method of operating an electrochemical gas sensor, comprising:
electrically coupling a switching circuit to an electrochemical cell that comprises a first electrode, a second electrode, and a third electrode;
electrically coupling a drive circuit to the switching circuit, the drive circuit including a working-electrode terminal, a counter-electrode terminal, and a reference-electrode terminal;
placing the switching circuit in a first state in which the first electrode is electrically coupled to the working-electrode terminal, the second electrode is electrically coupled to the counter-electrode terminal, and the third electrode is electrically coupled to the reference-electrode terminal; and
placing the switching circuit in a second state in which the second electrode is electrically coupled to the working-electrode terminal, the first electrode is electrically coupled to the counter-electrode terminal, and the third electrode is electrically coupled to the reference-electrode terminal.

35. The method of claim 34, further comprising:
when the switching circuit is in the first state, determining, with a computer in electrical communication with the drive circuit, a concentration of a first gas in an environment of the electrochemical gas sensor, the computer including a microprocessor and memory that is operatively coupled to the microprocessor; and
when the switching circuit is in the second state, determining, with the computer, a concentration of the second gas in the environment.

36. The method of claim 34, further comprising:
filtering ambient gases with a first filter that only allows one or more first gas(es) to pass through to the first electrode; and
when the switching circuit is in the first state, determining, with a computer in electrical communication with the drive circuit, a total concentration of the one or more first gas(es) in an environment of the electrochemical gas sensor, the computer including a microprocessor and memory that is operatively coupled to the microprocessor.

37. The method of claim 36, further comprising:
when the switching circuit is in the second state, determining with the computer, a state of the electrochemical cell; and
with the computer, adjusting a determination of the total concentration of the one or more first gas(es) based, at least in part, on the state of the electrochemical cell.

38. The method of claim 34, further comprising:
filtering ambient gases with a first filter that only allows first and second gases to pass through to the first electrode;
filtering the ambient gases with a second filter that only allows the first gas to pass through to the second electrode;
when the switching circuit is in the first state, determining, with a computer in electrical communication with the drive circuit, a total concentration of the first and second gases in an environment of the electrochemical gas sensor, the computer including a microprocessor and memory that is operatively coupled to the microprocessor; and
when the switching circuit is in the second state, determining, with the computer, a concentration of the first gas in the environment.

39. The method of claim 38, further comprising determining, with the computer, a concentration of the second gas in the environment based on the total concentration of the first and second gases and the concentration of the first gas.

40. The method of claim 39, wherein the first gas comprises nitrogen dioxide and the second gas comprises carbon monoxide.

41. The method of claim 34, further comprising:
filtering ambient gases with a first filter that only allows inorganic gases to pass through to the first electrode;
when the switching circuit is in the first state, determining, with a computer in electrical communication with the drive circuit, a concentration of the inorganic gases in an environment of the electrochemical gas sensor, the computer including a microprocessor and memory that is operatively coupled to the microprocessor;
when the switching circuit is in the second state, determining, with the computer, a total concentration of the organic and inorganic gases in the environment; and
determining, with the computer, a concentration of the organic gases in the environment based on the concentration of the inorganic gases and the total concentration of the organic and inorganic gases.

42. The method of claim 34, further comprising:
filtering ambient gases with a first filter that only allows organic gases to pass through to the first electrode;
when the switching circuit is in the first state, determining, with a computer in electrical communication with the drive circuit, a concentration of the organic gases in an environment of the electrochemical gas sensor, the computer including a microprocessor and memory that is operatively coupled to the microprocessor;
when the switching circuit is in the second state, determining, with the computer, a total concentration of the organic and inorganic gases in the environment; and
determining, with the computer, a concentration of the inorganic gases in the environment based on the concentration of the organic gases and the total concentration of the organic and inorganic gases.

43. A method of sensing a gas, comprising:
electrically coupling a first drive circuit to a first group of electrodes in an electrochemical cell;
electrically coupling a second drive circuit to a second group of electrodes in the electrochemical cell;
using the first drive circuit and the first group of electrodes to take a first gas measurement of an environment of the electrochemical cell; and
using the second drive circuit and the second group of electrodes to take a second gas measurement of the environment.

44. The method of claim 43, wherein the first and second groups of electrodes are the same.

45. The method of claim 43, wherein the first group of electrodes is different than the second group of electrodes.

46. The method of claim 45, wherein the first and second group electrodes do not include a common electrode.

47. The method of claim 43, further comprising:
operating the first drive circuit at a first frequency; and
operating the second drive circuit at a second frequency that is lower than the first frequency.

48. The method of claim 47, further comprising:
operating the first drive circuit at a first frequency range that includes the first frequency; and
performing electrochemical impedance spectroscopy over the first frequency range.

49. The method of claim 48, wherein:
the first frequency range comprises about 1 kHz to about 1 MHz, and the second frequency range comprises about 0 kHz to about 1 kHz.

50. The method of claim 43, further comprising determining, in a computer comprising a microprocessor, a composition of the gas using the first gas measurement and/or the second gas measurement.

51. The method of claim 50, further comprising:
receiving, at the computer, environment data from an external data source;
using the environment data to determine, in the computer, the composition of the gas and/or a concentration of the gas in the environment; and
generating an output signal, in the computer, that corresponds to the composition of the gas and/or the concentration of the gas.

52. The method of claim 51, wherein the environment data comprises a temperature of the environment, a relative humidity of the environment, an atmospheric pressure of the environment, and/or geolocation data of the computer.

53. A method of sensing a gas, comprising:
electrically coupling a first drive circuit to a group of electrodes in an electrochemical cell;
using the first drive circuit and the group of electrodes to take a gas measurement of an environment of the electrochemical cell;
electrically disconnecting the group of electrodes from the first drive circuit;
electrically coupling a second drive circuit to the group of electrodes;
using the second drive circuit and the group of electrodes to determine a state of the electrochemical cell; and
determining, with a computer in electrical communication with the first and second drive circuits, a composition and/or a concentration of the gas in the environment of the electrochemical cell using the gas measurement and the state of the electrochemical cell.

54. The method of claim 53, wherein the state of the electrochemical cell comprises an impedance between two electrodes in the group of electrodes.
